(19) United States
(12) Patent Application Publication
(10) Pub. No.: US 2011/0269777 A1
(43) Pub. Date: Nov. 3, 2011

(54) METHODS AND COMPOSITIONS FOR TREATING SCHIZOPHRENIA USING ANTIPSYCHOTIC COMBINATION THERAPY

(75) Inventors: Sergey O. Bachurin, Chernogolovka (RU); Vladimir V. Grigoriev, Chernogolovka (RU); Margarita A. Morozova, Chernogolovka (RU); Allan G. Benia, Beniashvili (RU)

(73) Assignee: Medivation Neurology, Inc.

(21) Appl. No.: 12/671,679

(22) PCT Filed: Aug. 1, 2008
(86) PCT No.: PCT/US08/09357

§ 371 Date: Apr. 28, 2011

(30) Foreign Application Priority Data
Aug. 1, 2007 (RU) 2007129567
Aug. 1, 2007 (RU) 2007129568

(51) Int. Cl. A61K 31/444; A61P 25/18; A61K 31/519

(52) U.S. Cl. 514/259.41; 514/292

(57) ABSTRACT

The present invention relates to combination therapies and methods for treating, preventing and/or delaying the onset and/or development of schizophrenia, wherein the combination therapies comprise a hydrogenated pyrido[4,3-b]indole or a pharmaceutically acceptable salt thereof, such as dimebon, and an antipsychotic.

### CROSS-REFERENCE TO RELATED APPLICATIONS

This application claims priority under the Paris Convention to Russian Patent Application No. 2007-129567, filed with the Russian Patent Office on Aug. 1, 2007, and to Russian Patent Application No. 2007-129568, filed with the Russian Patent Office on Aug. 1, 2007, both of which are incorporated herein by reference in their entirety.

### STATEMENT OF RIGHTS TO INVENTIONS MADE UNDER FEDERALLY SPONSORED RESEARCH

Not applicable.

### TECHNICAL FIELD

The invention relates to the field of medicine, and more specifically, to application of chemical compounds for the purpose of creating novel combination therapies and methods for treating, preventing and/or delaying the onset and/or development of schizophrenia.

### BACKGROUND OF THE INVENTION

### Summary of Schizophrenia

Schizophrenia dramatically affects the health and well-being of individuals who suffer from this mental disorder, which is among the most severe and difficult to treat. Individuals with schizophrenia (“schizophrenics”) can suffer from a myriad of symptoms and may require significant custodial care and continuous drug and/or behavior therapy, leading to substantial social and economic costs, even in the absence of hospitalization or institutionalization. Schizophrenia affects approximately 2 million Americans. The illness usually develops between adolescence and age 30 and is characterized by one or more positive symptoms (e.g., delusions and hallucinations) and/or negative symptoms (e.g., blunted emotions and lack of interest) and/or disorganized symptoms (e.g., confused thinking and speech or disorganized behavior and perception). Schizophrenics have been demonstrated in many studies to have degraded abilities at tasks requiring short-term verbal working memory (the ability to store and manipulate verbally presented information), rapidly associated cognitive “prediction” or “expectation,” ongoing attention/vigilance control and executive function (the ability to reason abstractly, plan, and solve problems). Schizophrenics who have auditory hallucinations (which describes the majority of afflicted individuals) also have a strongly correlated degradation in their speech reception abilities. Schizophrenics also have social and functional skill deficits, e.g., deficits and confusion in identifying the moods or reactions of others, in determining what for them is a socially correct course of action and in identifying the sources of current and past actions or events. Schizophrenia is a chronic disorder and most patients require constant treatment to alleviate or decrease the incidence of psychotic episodes. Although positive (psychotic and disorganized) symptoms may be most apparent to a lay observer, it is the negative symptoms and cognitive impairment of schizophrenia that correlate most highly with the inability to function effectively in society. The causes of schizophrenia are largely unknown. Although it is believed to have a genetic component, environmental factors appear to influence the onset and severity of the disease.

### Summary of Mechanistic Considerations in the Pathogenesis of Schizophrenia

Until recently, the attention of researchers working in the field of the biochemistry of psychoses was mainly concentrated on two mediator systems: the dopamine system and the serotonin system.

The dopamine hypothesis originated from the common ability of traditional (typical) antipsychotic drugs to cause neurological side effects similar to the symptoms of Parkinson's disease. This same property also gave the drugs the common name neuroleptics. The neurobiochemistry of Parkinsonism is connected with disruption of the balance between the dopaminergic and cholinergic systems in the nigrostriatum, in which the activity of the dopaminergic structures decreases, while the activity of the cholinergic structures increases. The ability of typical neuroleptics to control productive (psychotic) symptomatology in patients suffering from schizophrenic disorder (delusions, hallucinations, behavioral confusion) correlates with the ability to cause Parkinsonism and results from the property of suppressing the activity of the dopaminergic system. Thus, it was concluded that positive symptomatology of a psychosis is due to excessive activity of the dopaminergic system. One more argument in favor of this finding was the result of investigating dopamine metabolites in the spinal fluid. Higher levels of homovanilic acid (a product of dopamine metabolism) were found in psychotic patients than in healthy people. Currently this hypothesis has been developed further under the influence of new data involving the results of post-mortem examinations of the brain and positron emission tomography of living patients. The important regulator role of dopamine receptors was revealed by close study of the changes of function of the dopaminergic system under the effect of neuroleptic drugs. Several types of dopamine receptors have been described, each of which has its own features of localization and function.

Dopaminergic agents, first of all dopamine receptor subtype D₂ blockers, in particular, haloperidol and chlorpromazine and many others, are widely used for treatment of schizophrenia patients in accordance with the dopamine theory of schizophrenia. They effectively relieve the phase of acute psychosis in schizophrenia patients, but are often much less effective in the treatment of other phases of this disease. For this reason there has been intensive research to study the mechanism of the pathogenesis of schizophrenia and to develop new drugs for effective treatment of it in recent years.

The second hypothesis assumes that the fundamental cause is disruption in the relationship between the dopamine and serotonin systems. The serotoninergic structures carry out a complex modulating effect on the function of the dopaminergic system by increasing its activity in the mesolimbic and mesostriatal structures and reducing it in the prefrontal region, conditioning clinical hypofrontal function phenomena. A weighty argument for this hypothesis is usually considered to be the introduction of the prototype of atypical antipsychotics, clozapine, into clinical practice. The neurochemical spectrum of activity of clozapine distinguished it from all of the neuroleptics known at that time, since clozapine blocked serotoninergic receptors substantially more strongly than dopaminergic receptors. In addition, it proved to be effective with respect to illnesses where primary deficit disorders predominated and also in most cases that exhibited resistance to traditional neuroleptics. Moreover, clozapine caused neuroleptic side effects significantly less often. J. M. Kane, “The new antipsychotics,” J. Pract. Psychiatry Behav. Health, 1997, 3:343-354.

Data obtained in the course of clinical study of second-generation antipsychotics (serotonin-dopamine blockers—the so-called atypical antipsychotics “AA”) provide evidence of the superiority of these drugs over the neuroleptics of the first generation (dopamine blockers “DB”) in their effect on negative symptoms of schizophrenia, on resistant productive symptoms (i.e., delusions, hallucinations, and behavioral confusion), and neurocognitive disorders. Today there are a number of hypotheses, within the frameworks of which attempts are being made to explain the pharmacodynamic mechanisms that result in the superiority of AAs over first-generation neuroleptics (hypothesis of the predominant effect on the serotonin structures of suture nuclei, hypothesis of fast non-adhesive blockade of dopamine receptors, hypothesis of glutamate effects of clozapine). Bioclinical studies in the field of schizophrenia, including one due to the successes of psychopharmacology, are finding ever more convincing facts about the relationship between the development and persistence of clinical symptoms and neurocognitive disorders in cases of schizophrenia and a number of neurochemical, neuroimmunological, biochemical, genetic and morphological characteristics.

The hypotheses described above have sufficient explanatory power with respect to a large body of facts. However, not all data fit into them. It is known that the blockade of dopaminergic receptors occurs much faster than the clinical effect develops. In addition, the degree of blockade of these receptors is the same in patients who react well to antipsychotic therapy and patients who are resistant to it (S. Heckers, “Neural models of schizophrenia,” Dialogues in Clinical Neuroscience, 2000, 2(3): 267-280). On the other hand, the attempts of psychopharmacologists to develop a drug with antipsychotic effects that does not affect the dopaminergic system still have not led to success (S. Kapur, G. Remington, “Dopamine D(2) receptors and their role in atypical antipsychotic action: still necessary and may even be sufficient,” Biol. Psychiatry, 2001, 50 (11):873-83).

At the same time, not all practitioners see the change of generations of antipsychotic drugs the same way. Moreover, some take a skeptical view of the idea that second-generation drugs have a broader spectrum of efficacy. Indeed, studies in which the therapeutic response to a first-generation drug is compared to that of a second-generation drug do not show significant advantage in controlling productive symptoms of psychosis (i.e., delusions, hallucinations, and behavioral confusion). It is this effect in particular that is the traditional indicator of the therapeutic activity of an antipsychotic agent. A broadening of the notion of the pharmacodynamics of antipsychotic agents and of the possible reserve hidden in the remission that is achieved with typical therapy may be very important for a reconsideration of the attitudes of practicing psychiatrists toward new drugs.

Besides the widely recognized importance of the dopaminergic and serotoninergic activity of antipsychotic agents for the realization of their clinical activity, one more neuromediator system draws attention to itself. This is the glutamatergic neuromediator system of the central nervous system (CNS). Since many researchers in recent years have tended toward the opinion that cognitive disruptions play a fundamental role in the formation of schizophrenic disorder (N. C. Andreasen, “Schizophrenia: the fundamental questions,” Brain Res. Rev., 2000, 31(2-3):106-12), the glutamatergic system is causing ever growing interest, not only theoretically, but also practically (K. Hashimoto, M. Iyo, “Glutamate hypothesis of schizophrenia and targets for new antipsychotic drugs,” Nihon Shinkei Seishin Yakurigaku Zasshi, 2002, 22 (1):3-13). Stimulation of glutamatergic transmission can lead to stimulation of the activity of the central nervous system, but at some point it can also lead to toxic effects for the brain. On the other hand, depression of the glutamatergic system can lead to neuroprotector effects, but along with them, to a cognitive deficit (S. Heckers, C. Konradi, “Hippocampal neurons in schizophrenia,” J. Neural Transm., 2002, 109(5-6):891-905). Some researchers are proposing the ability to produce a glutamatergic effect as one possible neurochemical mechanism of the antideficit activity of clozapine (L. Chen, C. R. Yang, “Interaction of dopamine D1 and NMDA receptors mediates acute clozapine potentiation of glutamate EPSPs in rat prefrontal cortex,” J. Neurophysiol, 2002, 87(5):2324-36). In addition, the glutamatergic system is ascribed the role of coordinating the function of other mediator structures of the brain. This function can be implemented, in particular, due to the hypothetical ability of the cerebellum (in the functioning of which the glutamergic system plays an important role) to form temporary organization of mental processes (N. C. Andreasen, “Schizophrenia: the fundamental questions,” Brain Res. Rev. 2000, 31(2-3):106-12). Control of this function is hardly achievable for traditional antipsychotic drugs. However, the glutamate activity of clozapine in this connection yields an opportunity for the formation of new hypotheses that explain its unusual clinical activity over a long course of treatment (L. Chen, C. R. Yang, “Interaction of dopamine D1 and NMDA receptors mediates acute clozapine potentiation of glutamate EPSPs in rat prefrontal cortex,” J. Neurophysiol, 2002; 87(5):2324-36), and the formation of new homeostatic relationships requiring a long period of time. In spite of the instantaneous blockade of dopamine receptors, the first signs of the clinical effect of antipsychotics (control of productive symptoms) are realized gradually, over several weeks, and the improvement of the patients' conditions lasts many months.

Thus, along with the theory of the pathogenesis of schizophrenia that was developed a relatively long while ago and that is widely accepted, where the main role is given to hyperfunctioning of the dopaminergic neuromediator system of the CNS and also to imbalance in the serotoninergic mediator system, very recently there has been intensive development of a theory of pathogenesis where the main role in the development of this disease is played by disruptions in the glutamatergic neuromediator system of the CNS. It is proposed that many elements of psychic disorder that are observed in schizophrenia patients are connected with hypofunctioning of the glutamatergic system. Support for the glutamate theory of schizophrenia include the fact that phencyclidine, a blocker of the NMDA receptor ion channel, one of the principal subtypes of glutamate receptors, causes a complex of behavioral symptoms that are very similar to the behavior of schizophrenia patients in healthy volunteers: they exhibit alienation, autism, negative mood; they become unable to solve cognition problems (tests); they grow eccentric and their speech and thinking become impoverished. Currently, the phencyclidine model of schizophrenia is considered to be the closest and most adequate to the behavior of schizophrenia patients (R. M. Allen, S. J. Young, “Phencyclidine-induced psychosis,” Amer. J. Psych., 1976, 33:1425-8). Similar effects are also caused by other NMDA receptor ion channel blockers such as ketamine and AMPA blockers such as MK-801. It has been shown that schizophrenia patients exhibit a lower level of glutaminic acid in the cerebrospinal fluid than normal people. It has also been shown in subsequent studies that the brain of schizophrenia patients shows an increase of large diameter glutamatergic fibers that is 30% over that in the brain of patients not suffering from schizophrenia and that there is a simultaneous decrease of small diameter glutamatergic fibers by 78%. In addition, an increase of the number of NMDA receptors is seen in the cerebral cortex in schizophrenia patients, but there is also a decrease of the reverse capture of glutamate in basal ganglia.

### Summary of Hydrogenated Pyrido[4,3-b]Indole Derivatives

Known compounds of the class of tetra- and hexahydro-1H-pyrido[4,3-b]indole derivatives manifest a broad spectrum of biological activity. In the series of 2,3,4,5-tetrahydro-1H-pyrido[4,3-b]indoles the following types of activity have been found: antihistamine activity (DE 1,813,229, filed Dec. 6, 1968; DE 1,952,800, filed Oct. 20, 1969), central depressive and anti-inflammatory activity (U.S. Pat. No. 3,718,657, filed Dec. 3, 1970), neuroleptic activity (Herbert C. A., Plattner S. S., Welch W. M., Mol. Pharm. 1980, v. 17, N 1, p. 38-42) and others. 2,3,4,4a,5,9b-hexahydro-1H-pyrido[4,3-b]indole derivatives show psychotropic (Welch W. M., Herbert C. A., Weissman A., Koe B. K., J. Med. Chem., 1986, Vol. 29, No. 10, p. 2093-2099), antiaggressive, antiarrhythmic and other types of activity.

Several drugs, such as diazoline (mebhydroline), dimebon, dorastine, carbidine (dicarbine), stobadine and gevotroline, based on tetra- or hexahydro-1H-pyrido[4,3-b]indole derivatives are known to have been manufactured. Diazoline (2-methyl-5-benzyl-2,3,4,5-tetrahydro-1H-pyrido[4,3-b]indole dihydrochloride) (Klyuev M. A., Drugs, used in “Medical Pract.”, USSR, Moscow, “Meditzina” Publishers, 1991, p. 512) and dimebon (2,8-dimethyl-5-(2-(6-methyl-3-pyridyl)ethyl-2,3,4,5-tetrahydro-1H-pyrido[4,3-b]indole dihydrochloride) (M. D. Mashkovsky, “Medicinal Drugs” in 2 vol. Vol. 1, 12th Edition, Moscow, “Meditzina” Publishers, 1993, p. 383) as well as dorastine (2-methyl-8-chloro-5-[2-(6-methyl-3-pyridyl)ethyl]-2,3,4,5-tetrahydro-1H-pyrido[4,3-b]indole dihydrochloride) (USAN and USP dictionary of drugs names (United States Adopted Names, 1961-1988, current US Pharmacopoeia and National Formula for Drugs and other nonproprietary drug names), 1989, 26th. Ed., p. 196) are known as antihistamine drugs; carbidine (dicarbine) (cis(±)-2,8-dimethyl-2,3,4,4a,5,9b-hexahydro-1H-pyrido[4,3-b]indole dihydrochloride) is a neuroleptic agent having an antidepressive effect (L. N. Yakhontov, R. G. Glushkov, Synthetic Drugs, ed. by A. G. Natradze, Moscow, “Meditzina” Publishers, 1983, p. 234-237), and its (−)isomer, stobadine, is known as an antiarrythmic agent (Kitlova M., Gibela P., Drimal J., Bratisl. Lek. Listy, 1985, vol. 84, No. 5, p. 542-549); gevotroline 8-fluoro-2-(3-(3-pyridyl)propyl)-2,3,4,5-tetrahydro-1H-pyrido[4,3-b]indole dihydrochloride is an antipsychotic and anxiolytic agent (Abou-Gharbi M., Patel U. R., Webb M. B., Moyer J. A., Ardnee T. H., J. Med. Chem., 1987, vol. 30, p. 1818-1823). Dimebon has been used in medicine as an antiallergic agent (Inventor's Certificate No. 1138164, IP Class A61K 31/47,5, C07 D 209/52, published on Feb. 7, 1985) in Russia for over 20 years.

As described in U.S. Pat. Nos. 6,187,785 and 7,071,206, hydrogenated pyrido[4,3-b]indole derivatives, such as dimebon, have NMDA antagonist properties, which make them useful for treating neurodegenerative diseases, such as Alzheimer's disease. As described in WO 2005/055951, hydrogenated pyrido[4,3-b]indole derivatives, such as dimebon, are useful as human or veterinary geroprotectors e.g., by delaying the onset and/or development of an age-associated or related manifestation and/or pathology or condition, including disturbance in skin-hair integument, vision disturbance and weight loss. As described in WO 2007/087425, hydrogenated pyrido[4,3-b]indole derivatives, such as dimebon, are useful for treating and/or preventing and/or delaying the onset and/or the development of schizophrenia. U.S. patent application Ser. No. 11/543,529 (U.S. Publication No. 2007/0117835 A1) and Ser. No. 11/543,341 (U.S. Publication No. 2007/0117834 A1) disclose hydrogenated pyrido[4,3-b]indole derivatives, such as dimebon, as neuroprotectors for use in treating and/or preventing and/or slowing the progression or onset and/or development of Huntington's disease. Dimebon and/or its properties are also discussed in: Yu. Ya. Ivanov et al., 2001; N. N. Lermontova et al., 2001; S. O. Bachurin et al., 2003 and V. V. Grigor'ev et al., 2003.

### Significant Medical Need

There remains a significant interest in and need for additional or alternative therapies for treating, preventing and/or delaying the onset and/or development of schizophrenia, including its positive (productive), negative (deficit), and/or cognitive aspects. Preferably, new therapies improve the quality of life for patients with schizophrenia and/or are accompanied by fewer or less severe side effects as compared to currently available therapies.

### BRIEF SUMMARY OF THE INVENTION

Methods, combination therapies, pharmaceutical compositions and kits for treating and/or preventing and/or delaying the onset and/or the development of schizophrenia (including its positive, negative, and/or cognitive aspects) using a hydrogenated[4,3-b]indole and an antipsychotic or pharmaceutically acceptable salt of any of the foregoing are described. The invention embraces combination therapies having a first compound and a second agent, where the first compound is a hydrogenated[4,3-b]indole detailed herein and the second agent is an antipsychotic. The second agent may be either a typical antipsychotic or an atypical antipsychotic or a combination of an atypical and a typical antipsychotic (in which case the second agent could contain at least two different compounds). The invention particularly embraces a combination therapy wherein the first compound is dimebon (2,8-dimethyl-5-(2-(6-methyl-3-pyridyl)ethyl)-2,3,4,5-tetrahydro-1H-pyrido[4,3-b]indole dihydrochloride) and the second agent is an atypical antipsychotic, including but not restricted to risperidone (3-[2-[4-(6-fluoro-1,2-benzoxazol-3-yl)piperidin-1-yl]ethyl]-2-methyl-6,7,8,9-tetrahydropyrido[2,1-b]pyrimidin-4-one) and/or a typical antipsychotic, in particular perphenazine, or a pharmaceutically acceptable salt of any of the foregoing. In one variation, the antipsychotic component of the combination therapy is not an atypical antipsychotic.

In various embodiments, the invention embraces a method of (a) treating schizophrenia (including its positive, negative, and/or cognitive aspects) in an individual in need thereof; (b) slowing the progression of schizophrenia in an individual who has been diagnosed with schizophrenia; or (c) preventing or delaying development of schizophrenia in an individual who is at risk of developing schizophrenia, the method comprising administering to the individual an effective amount of a combination therapy comprising dimebon and an antipsychotic. In one variation, the methods of the invention employ a combination therapy whereby the antipsychotic is other than an atypical antipsychotic. In one variation, the antipsychotic is an atypical antipsychotic. In one variation, the atypical antipsychotic is selected from the group consisting of risperidone, clozapine, N-desmethylclozapine, olanzapine, quetiapine, perospirone, ziprasidone, olanzapine/fluoxetine (marketed as Symbyax™), aripiprazole, paliperidone, sertindole, zotepine, amisulpride, bifeprunox, asenapine, melperone, abaperidone, blonanserin, iloperidone, lurasidone, ocaperidone, QF-2400B, SB-773812, ITI-007, and YKP-1358. In one variation, the atypical antipsychotic is risperidone. In one variation, the antipsychotic is a typical antipsychotic. In one variation, the typical antipsychotic is selected from the group consisting of chlorpromazine, trifluoroperazine hydrochloride, fluphenazine HCl or fluphenazine decanoate, haloperidol, molindone, thiothixene, thioridazine, trifluoperazine, loxapine, perphenazine, prochlorperazine, pimozide, and zuclopenthixol. In one variation, the typical antipsychotic is perphenazine. In one variation, the antipsychotic is a combination of an atypical antipsychotic and a typical antipsychotic (in which case the second agent could contain at least two different compounds). In one variation, the antipsychotic is a combination of an atypical antipsychotic selected from the group consisting of risperidone, clozapine, N-desmethylclozapine, olanzapine, quetiapine, perospirone, ziprasidone, olanzapine/fluoxetine (marketed as Symbyax™), aripiprazole, paliperidone, sertindole, zotepine, amisulpride, bifeprunox, asenapine, melperone, abaperidone, blonanserin, iloperidone, lurasidone, ocaperidone, QF-2400B, SB-773812, ITI-007, and YKP-1358, and a typical antipsychotic selected from the group consisting of chlorpromazine, trifluoroperazine hydrochloride, fluphenazine HCl or fluphenazine decanoate, haloperidol, molindone, thiothixene, thioridazine, trifluoperazine, loxapine, perphenazine, prochlorperazine, pimozide, and zuclopenthixol. In one variation, the antipsychotic is a combination of the atypical antipsychotic risperidone and the typical antipsychotic perphenazine.

In one variation, the method is a method of alleviating one or more positive symptoms of schizophrenia by administering to an individual an effective amount of a combination therapy. In one variation, the method is a method of alleviating one or more negative symptoms of schizophrenia by administering to an individual an effective amount of a combination therapy. In one variation, the method is a method of alleviating one or more cognitive symptoms of schizophrenia by administering to an individual an effective amount of a combination therapy. In one variation, the method is a method of alleviating one or more disorganized symptoms of schizophrenia by administering to an individual an effective amount of the combination therapy. In any of the above variations, the methods of the invention employ a combination therapy whereby the antipsychotic is other than an atypical antipsychotic. In any of the above variations, the antipsychotic is an atypical antipsychotic. In any of the above variations, the atypical antipsychotic is selected from the group consisting of risperidone, clozapine, N-desmethylclozapine, olanzapine, quetiapine, perospirone, ziprasidone, olanzapine/fluoxetine (marketed as Symbyax™), aripiprazole, paliperidone, sertindole, zotepine, amisulpride, bifeprunox, asenapine, melperone, abaperidone, blonanserin, iloperidone, lurasidone, ocaperidone, QF-2400B, SB-773812, ITI-007, and YKP-1358. In any of the above variations, the atypical antipsychotic is risperidone. In any of the above variations, the antipsychotic is a typical antipsychotic. In any of the above variations, the typical antipsychotic is selected from the group consisting of chlorpromazine, trifluoroperazine hydrochloride, fluphenazine HCl or fluphenazine decanoate, haloperidol, molindone, thiothixene, thioridazine, trifluoperazine, loxapine, perphenazine, prochlorperazine, pimozide, and zuclopenthixol. In any of the above variations, the typical antipsychotic is perphenazine. In any of the above variations, the antipsychotic is a combination of an atypical antipsychotic and a typical antipsychotic (in which case the second agent could contain at least two different compounds). In any of the above variations, the antipsychotic is a combination of an atypical antipsychotic selected from the group consisting of risperidone, clozapine, N-desmethylclozapine, olanzapine, quetiapine, perospirone, ziprasidone, olanzapine/fluoxetine (marketed as Symbyax™), aripiprazole, paliperidone, sertindole, zotepine, amisulpride, bifeprunox, asenapine, melperone, abaperidone, blonanserin, iloperidone, lurasidone, ocaperidone, QF-2400B, SB-773812, ITI-007, and YKP-1358, and a typical antipsychotic selected from the group consisting of chlorpromazine, trifluoroperazine hydrochloride, fluphenazine HCl or fluphenazine decanoate, haloperidol, molindone, thiothixene, thioridazine, trifluoperazine, loxapine, perphenazine, prochlorperazine, pimozide, and zuclopenthixol. In any of the above variations, the antipsychotic is a combination of the atypical antipsychotic risperidone and the typical antipsychotic perphenazine.

In one variation, the method is a method of alleviating one or more symptoms of schizophrenia by administering to an individual an effective amount of the combination therapy. In one variation, the method is a method of alleviating a positive and a negative symptom of schizophrenia by administering to an individual an effective amount of the combination therapy. In another variation, the method is a method of alleviating a positive and a disorganized symptom of schizophrenia. In yet another variation, the method is a method of alleviating a negative and a disorganized symptom of schizophrenia by administering to an individual an effective amount of the combination therapy. In yet another variation, the method is a method of alleviating a positive and/or a cognitive symptom of schizophrenia by administering to an individual an effective amount of the combination therapy. In another variation, the method is a method of alleviating a negative and/or a cognitive symptom of schizophrenia by administering to an individual an effective amount of the combination therapy. In yet another variation, the method is a method of alleviating a disorganized and/or a cognitive symptom of schizophrenia by administering to an individual an effective amount of the combination therapy. In another variation, the method is a method of alleviating a positive, a negative and a disorganized symptom of schizophrenia by administering to an individual an effective amount of the combination therapy. In yet another variation, the method is a method of alleviating a positive, a negative and/or a cognitive symptom of schizophrenia by administering to an individual an effective amount of the combination therapy. In another variation, the method is a method of alleviating a negative, a disorganized and/or a cognitive symptom of schizophrenia by administering to an individual an effective amount of the combination therapy. In yet another variation, the method is a method of alleviating a positive, a negative, a disorganized and/or a cognitive symptom of schizophrenia by administering to an individual an effective amount of the combination therapy. In any of the above variations, the methods of the invention employ a combination therapy whereby the antipsychotic is other than an atypical antipsychotic. In any of the above variations, the antipsychotic is an atypical antipsychotic. In any of the above variations, the atypical antipsychotic is selected from the group consisting of risperidone, clozapine, N-desmethylclozapine, olanzapine, quetiapine, perospirone, ziprasidone, olanzapine/fluoxetine (marketed as Symbyax™), aripiprazole, paliperidone, sertindole, zotepine, amisulpride, bifeprunox, asenapine, melperone, abaperidone, blonanserin, iloperidone, lurasidone, ocaperidone, QF-2400B, SB-773812, ITI-007, and YKP-1358. In any of the above variations, the atypical antipsychotic is risperidone. In any of the above variations, the antipsychotic is a typical antipsychotic. In any of the above variations, the typical antipsychotic is selected from the group consisting of chlorpromazine, trifluoroperazine hydrochloride, fluphenazine HCl or fluphenazine decanoate, haloperidol, molindone, thiothixene, thioridazine, trifluoperazine, loxapine, perphenazine, prochlorperazine, pimozide, and zuclopenthixol. In any of the above variations, the typical antipsychotic is perphenazine. In any of the above variations, the antipsychotic is a combination of an atypical antipsychotic and a typical antipsychotic (in which case the second agent could contain at least two different compounds). In any of the above variations, the antipsychotic is a combination of an atypical antipsychotic selected from the group consisting of risperidone, clozapine, N-desmethylclozapine, olanzapine, quetiapine, perospirone, ziprasidone, olanzapine/fluoxetine (marketed as Symbyax™); aripiprazole, paliperidone, sertindole, zotepine, amisulpride, bifeprunox, asenapine, melperone, abaperidone, blonanserin, iloperidone, lurasidone, ocaperidone, QF-2400B, SB-773812, ITI-007, and YKP-1358, and a typical antipsychotic selected from the group consisting of chlorpromazine, trifluoroperazine hydrochloride, fluphenazine HCl or fluphenazine decanoate, haloperidol, molindone, thiothixene, thioridazine, trifluoperazine, loxapine, perphenazine, prochlorperazine, pimozide, and zuclopenthixol. In any of the above variations, the antipsychotic is a combination of the atypical antipsychotic risperidone and the typical antipsychotic perphenazine.

In any of the above variations, an antipsychotic of a combination therapy is administered in a dosage that is less than that required for the same antipsychotic monotherapy (or dual therapy where an atypical antipsychotic is administered in connection with a typical antipsychotic) to elicit a comparable therapeutic effect.

Also embraced by the invention are methods of enhancing an individual's response to an antipsychotic by administering a first compound such as dimebon in connection with the antipsychotic. The invention further includes methods of treating schizophrenia by administering a combination therapy comprising dimebon and an antipsychotic wherein the combination therapy is administered in an amount effective to improve a positive, a negative, and/or a cognitive symptom of schizophrenia. Particularly, the invention embraces combination therapies that elicit cognitive improvement in an individual. The invention embraces methods that enhance an individual's cognitive ability (improves cognition/lessens the number and/or severity of cognitive symptoms associated with schizophrenia) to a greater extent than use of an antipsychotic as an individual/monotherapy (and in the absence of a first compound such as dimebon) in the same or similar subjects.

The invention also embraces pharmaceutical compositions of the combination therapy, including unit dosage forms thereof. Where applicable to any of the embodiments described herein, such as any of the methods described herein, in one variation, the combination therapy employs an antipsychotic that is not an atypical antipsychotic. In one variation, the antipsychotic is an atypical antipsychotic. In one variation, the atypical antipsychotic is selected from the group consisting of risperidone, clozapine, N-desmethylclozapine, olanzapine, quetiapine, perospirone, ziprasidone, olanzapine/fluoxetine (marketed as Symbyax™), aripiprazole, paliperidone, sertindole, zotepine, amisulpride, bifeprunox, asenapine, melperone, abaperidone, blonanserin, iloperidone, lurasidone, ocaperidone, QF-2400B, SB-773812, ITI-007, and YKP-1358. In one variation, the atypical antipsychotic is risperidone. In one variation, the antipsychotic is a typical antipsychotic. In one variation, the typical antipsychotic is selected from the group consisting of chlorpromazine, trifluoroperazine hydrochloride, fluphenazine HCl or fluphenazine decanoate, haloperidol, molindone, thiothixene, thioridazine, trifluoperazine, loxapine, perphenazine, prochlorperazine, pimozide, and zuclopenthixol. In one variation, the typical antipsychotic is perphenazine. In one variation, the antipsychotic is a combination of an atypical antipsychotic and a typical antipsychotic (in which case the second agent could contain at least two different compounds). In one variation, the antipsychotic is a combination of an atypical antipsychotic selected from the group consisting of risperidone, clozapine, N-desmethylclozapine, olanzapine, quetiapine, perospirone, ziprasidone, olanzapine/fluoxetine (marketed as Symbyax™), aripiprazole, paliperidone, sertindole, zotepine, amisulpride, bifeprunox, asenapine, melperone, abaperidone, blonanserin, iloperidone, lurasidone, ocaperidone, QF-2400B, SB-773812, ITI-007, and YKP-1358, and a typical antipsychotic selected from the group consisting of chlorpromazine, trifluoroperazine hydrochloride, fluphenazine HCl or fluphenazine decanoate, haloperidol, molindone, thiothixene, thioridazine, trifluoperazine, loxapine, perphenazine, prochlorperazine, pimozide, and zuclopenthixol. In one variation, the antipsychotic is a combination of the atypical antipsychotic risperidone and the typical antipsychotic perphenazine.

### DETAILED DESCRIPTION OF THE INVENTION

Surprisingly, administration of a combination therapy comprising dimebon and the atypical antipsychotic risperidone to clinical trial participants having schizophrenia, paranoid type, chronic course resulted in a significant reduction in total Positive and Negative Symptom Scale (PANSS) scores relative to patients receiving risperidone alone (i.e., placebo). Analysis of the differences between patients receiving dimebon and placebo on PANSS negative change scores, in particular on the NSA-16, supports the utility of dimebon in treating negative symptoms of schizophrenia. Furthermore, the data also suggested a benefit on general cognitive symptoms. In particular, the dimebon group demonstrated significant improvement in verbal associative memory, psychomotor speed, visual-spatial memory and number aspects of executive functioning—planning, purposeful activity and control upon the results of activity (perseverative errors), as shown by the results of the Wechsler Memory Scale Subtest VII, the Text Reconstruction test, the Benton test, the Bourdohn test, and the Tower of London test (see, e.g., Table 3). The placebo-controlled, double-blind portion of the study lasted only eight weeks, which is considered short for a trial of a putative enhancer of cognition in schizophrenia patients. Thus, these results suggest the potential for dimebon to provide a cognitive benefit in this patient population when studied for a longer duration, particularly in the memory and executive function domains that are significantly affected in schizophrenia.

For use herein, unless clearly indicated otherwise, use of the terms “a”, “an” and the like refers to one or more.

Reference to “about” a value or parameter herein includes (and describes) embodiments that are directed to that value or parameter per se. For example, description referring to “about X” includes description of “X”.

As used herein, “the combination therapy” or “a combination therapy” is meant a therapy comprising a first compound and a second agent, wherein the first compound is a hydrogenated pyrido[4,3-b]indole as described herein and the second agent is an antipsychotic and where the first compound is used in conjunction with the second agent. A therapy comprising dimebon used in conjunction with risperidone is an example of a combination therapy according to the invention. Administration of a first compound “in conjunction with” a second agent includes administration of the compounds in the same or a different composition, either sequentially, simultaneously, or continuously. The term administration “in conjunction with” encompasses any circumstance wherein a first compound (such as dimebon) and a second agent (such as risperidone or perphenazine) are administered in an effective amount to an individual. As further discussed herein, it is understood that the first compound and the second agent can be administered at different dosing frequencies and/or intervals and may be administered using the same route of administration or different routes of administration. For instance, administration “in conjunction with” embraces a dosing regimen whereby a first compound of the combination therapy is administered three times daily and a second agent of the combination therapy is administered once daily and wherein the first daily dose of the first compound is administered simultaneously with the second agent and where the second and the third daily doses of the first compound are administered alone (in the absence of a second agent). It is further understood that different dosing regimens may change over the course of administration. For example, in a combination therapy comprising dimebon and risperidone, dimebon may be administered daily and risperidone may be administered weekly or less than daily. Alternatively, dimebon may be administered weekly or less than daily and risperidone may be administered daily. In some variations, the combination therapy optionally includes one or more pharmaceutically acceptable carriers or excipients, non-pharmaceutically active compounds, and/or inert substances. Thus, the compounds in a combination therapy of the invention may be administered sequentially, simultaneously, or continuously using the same or different routes of administration for each compound.

It is also understood and clearly conveyed by this disclosure that reference to “the first compound” or “a first compound” includes and refers to any hydrogenated pyrido[4,3-b]indole or pharmaceutically acceptable salt or other form thereof as described herein, such as the compound dimebon.

It is also understood and clearly conveyed by this disclosure that reference to “the second agent” or “a second agent” of a combination therapy includes and refers to an antipsychotic or pharmaceutically acceptable salt thereof. The second agent may be an atypical and/or a typical antipsychotic, or a combination of an atypical antipsychotic and a typical antipsychotic (in which case the second agent could contain at least two different compounds).

As used herein, the term “schizophrenia” includes all forms and classifications of schizophrenia known in the art, including, but not limited to catatonic type, hebephrenic type, disorganized type, paranoid type, residual type or undifferentiated type schizophrenia and deficit syndrome and/or those described in American Psychiatric Association: Diagnostic and Statistical Manual of Mental Disorders, Fourth Edition, Washington D.C., 2000 or in International Statistical Classification of Diseases and Related Health Problems, or otherwise known to those skilled in the art.

As used herein, the term “antipsychotic” refers to and encompasses an atypical and/or a typical antipsychotic. In one variation, the combination therapy employs an atypical antipsychotic. In one variation, the combination therapy employs a typical antipsychotic. In one variation, the combination therapy employs an atypical antipsychotic and a typical antipsychotic. In a particular variation, the combination therapy employs an antipsychotic other than an atypical antipsychotic (in one variation, an atypical antipsychotic is excluded).

As used herein, the term “atypical antipsychotic” intends an antipsychotic that reduces or eliminates an activity of a serotonin-2A (5-HT2A) receptor and a dopamine-2 (D2) receptor. In some embodiments, the atypical antipsychotic reduces an activity of a serotonin-2A (5-HT2A) receptor and a dopamine-2 (D2) receptor by at least or about any of 10%, 20%, 30%, 40%, 50%, 60%, 70%, 80%, 90%, 95% or 100% as compared to the corresponding activity in the same subject prior to treatment with the atypical antipsychotic or compared to the corresponding activity in other subjects not receiving the atypical antipsychotic. In some embodiments, the atypical antipsychotic is capable of binding to the active site of at least one of a 5-HT2A receptor and a D2 receptor (e.g., a binding site for a ligand). In some embodiments, the atypical antipsychotic is capable of binding to an allosteric site of at least one of a 5-HT2A receptor and a D2 receptor. The interaction between the atypical antipsychotic and a 5-HT2A receptor and a D2 receptor may be reversible or irreversible. In some embodiments, the atypical antipsychotic reduces the amount or extent of motor side effects, such as extrapyramidal side effects (EPS) [e.g., akathisia (an unpleasant sensation of restlessness that may be accompanied by overtly increased motor activity), dystonia (a movement disorder in which sustained muscle contractions cause twisting or repetitive movements or abnormal postures), and/or Parkinsonism (characterized by rigidity, bradykinesia, postural instability, resting tremor, masked faces and or shuffling gait)] and tardive dyskinesia (repetitive, involuntary, purposeless movements including but not limited to grimacing, lip smacking, tongue protrusion, and pursing of the lips), as compared to typical antipsychotics given to the same or other subjects at standard doses. Examples of atypical antipsychotics include, but are not limited to, risperidone (marketed as Risperdal™) (3-[2-[4-(6-fluoro-1,2-benzoxazol-3-yl)piperidin-1-yl]ethyl]-2-methyl-6,7,8,9-tetrahydropyrido[2,1-b]pyrimidin-4-one); clozapine (marketed as Clozaril™) (3-chloro-6-(4-methylpiperazin-1-yl)-5H-benzo[c][1,5]benzodiazepine); N-desmethylclozapine (also known as ACP-104, a major metabolite of clozapine; Acadia Pharmaceuticals; currently in Phase II clinical trials); olanzapine (marketed as Zyprexa™) (2-methyl-4-(4-methylpiperazin-1-yl)-5H-thieno[3,2-c][1,5]benzodiazepine); quetiapine (marketed as Seroquel™) (2-[2-(4-benzo[b][1,5]benzothiazepin-6-ylpiperazin-1-yl)ethoxy]ethanol); perospirone (cis-N-[4-[4-(1,2-benz-isozole-3-yl)-1-piperazinyl]butyl]cyclohexane-1,2-dicarboximide hydrochloride); ziprasidone (marketed as Geodon™) (5-[2-[4-(1,2-benzothiazol-3-yl)piperazin-1-yl]ethyl]-6-chloro-1,3-dihydroindol-2-one); olanzapine/fluoxetine (marketed as Symbyax™), aripiprazole (marketed as Abilify™) (7-[4-[4-(2,3-dichlorophenyl)piperazin-1-yl]butoxy]-3,4-dihydro-1H-quinolin-2-one; paliperidone (marketed as Invega™) (3-[2-[4-(6-fluorobenzo[d]isoxazol-3-yl)-1-piperidyl]ethyl]-7-hydroxy-4-methyl-1,5-diazabicyclo[4.4.0]deca-3,5-dien-2-one); sertindole (also known as Serlect™) (1-[2-[4-[5-chloro-1-(4-fluorophenyl)-indol-3-yl]-1-piperidyl]ethyl]imidazolidin-2-one); zotepine (2-((8-Chlorodibenzo(b,f)thiepin-10-yl)oxy)-N,N-dimethylethylamine); amisulpride (4-amino-N-[(1-ethylpyrrolidin-2-yl)methyl]-5-ethylsulfonyl-2-methoxy-benzamide); bifeprunox (7-[4-[(3-Phenylphenyl)methyl]piperazin-1-yl]-3H-benzooxazol-2-one); asenapine (trans-5-chloro-2-methyl-2,3,3a,12b-tetrahydro-1H-dibenz[2,3:6,7]oxepino[4,5-c]pyrrole); melperone (1-(4-fluorophenyl)-4-(4-methyl-1-piperidyl)butan-1-one); abaperidone (7-(3-(4-(6-fluoro-1,2-benzisoxazol-3-yl)piperidin-1-yl)propoxy)-3-hydroxymethyl)chromen-4-one); blonanserin (2-(4-ethyl-1-piperazinyl)-4-(4-fluorophenyl)-5,6,7,8,9,10-hexahydrocycloocta(b)pyridine); iloperidone (marketed as Zomaril™) (1-[4-[3-[4-(6-fluoro-1,2-benzisoxazol-3-yl)-1-piperidinyl]propoxy]-3-methoxyphenyl]ethanone); lurasidone (N-2-(4-(1,2-benzisothiazol-3-yl)-1-piperazinylmethyl)-1-cyclohexylmethyl)-2,3-bicyclo(2.2.1)heptanedicarboximide); ocaperidone (3-[2-[4-(6-fluoro-1,2-benzoxazol-3-yl)piperidin-1-yl]ethyl]-2,9-dimethylpyrido[2,1-b]pyrimidin-4-one); QF-2400B (2-[4-(6-fluorobenzisoxazol-3-yl)piperidinyl]methyl-1,2,3,4-tetrahydro-carbazol-4-one); SB-773812 (GlaxoSmithKline PLC; currently in Phase II clinical trials); ITI-007 (Intra-Cellular Therapies, Inc.; currently in Phase I clinical trials); and YKP-1358 (SK-BioPharmaceuticals; currently in Phase I clinical trials).

As used herein, the term “typical antipsychotic” intends an antipsychotic that reduces or eliminates primarily an activity of a dopamine-2 (D2) receptor in a reversible or irreversible manner. In some embodiments, the typical antipsychotic reduces an activity of a D2 receptor by at least or about any of 10%, 20%, 30%, 40%, 50%, 60%, 70%, 80%, 90%, 95% or 100% as compared to the corresponding activity in the same subject prior to treatment with the typical antipsychotic or compared to the corresponding activity in other subjects not receiving the typical antipsychotic. In some embodiments, the typical antipsychotic is capable of binding to the active site of a D2 receptor (e.g., a binding site for a ligand). In some embodiments, the typical antipsychotic is capable of binding to an allosteric site of a D2 receptor. Examples of typical antipsychotics include, but are not limited to, chlorpromazine (marketed as Largactil™ or Thorazine™) (3-(2-chloro-10H-phenothiazin-10-yl)-N,N-dimethyl-propan-1-amine); trifluoroperazine hydrochloride (10-[3-(4-methylpiperazin-1-yl)propyl]-2-(trifluoromethyl)phenothiazine); fluphenazine HCl or fluphenazine decanoate (marketed as Prolixin™ or Prolixin Decanoate™) (2-[4-[3-[2-(trifluoromethyl)-10H-phenothiazin-10-yl]propyl]-piperazin-1-yl]ethanol); haloperidol (marketed as Haldol™ or Serenace™) (4-[4-(4-chlorophenyl)-4-hydroxy-1-piperidyl]-1-(4-fluorophenyl)-butan-1-one); molindone (marketed as Moban™) (3-ethyl-2-methyl-5-(morpholin-4-ylmethyl)-1,5,6,7-tetrahydro-4H-indol-4-one); thiothixene (marketed as Navane™) ((Z)-N,N-dimethyl-9-[3-(4-methylpiperazin-1-yl)propylidene]-thioxanthene-2-sulfonamide); thioridazine (marketed as Mellaril™) (10-{2-[(RS)-1-Methylpiperidin-2-yl]ethyl}-2-methylsulfanyl-phenothiazine); trifluoperazine (marketed as Stelazine™) (10-[3-(4-methylpiperazin-1-yl)propyl]-2-(trifluoromethyl)-10H-phenothiazine); loxapine (marketed as Loxapac™ or Loxitane™) (2-Chloro-11-(4-methylpiperazin-1-yl)dibenzo[b,f][1,4]oxazepine); perphenazine (marketed as Trilafon™) (2-[4-[3-(2-chloro-10H-phenothiazin-10-yl)propyl]piperazin-1-yl]ethanol); prochlorperazine (marketed as Compazine™, Buccastem™, or Stematil™) (2-chloro-10-[3-(4-methyl-1-piperazinyl)propyl]-10H-phenothiazine); pimozide (marketed as Orap™) (1-[1-[4,4-bis(4-fluorophenyl)butyl]-4-piperidinyl]-1,3-dihydro-2H-benzimidazole-2-one); and zuclopenthixol (marketed as Clopixol Dihydrochloride™ or Clopixol Decanoate™) (cis(Z)-4-[3-(2-chlorothioxanthen-9-ylidene)propyl]-1-piperazineethanol).

As used herein, “treatment” or “treating” is an approach for obtaining a beneficial or desired result, including clinical results (e.g., reducing the severity or duration of, stabilizing the severity of, or eliminating one or more symptoms (biochemical, histological and/or behavioral) of schizophrenia). For purposes of this invention, beneficial or desired results include, but are not limited to, alleviation of symptoms associated with schizophrenia, diminishment of the extent of the symptoms associated with schizophrenia, preventing a worsening of the symptoms associated with schizophrenia, including positive and/or negative and/or disorganized symptoms. Treatment embraces increasing the quality of life of those suffering from schizophrenia, decreasing the dose of other medications required to treat schizophrenia, delaying the progression of schizophrenia and/or prolonging survival of schizophrenia patients. Preferably, treatment with a combination therapy disclosed herein, is accompanied by no or fewer side effects than those that are commonly associated with administration of antipsychotic drugs, such as extrapyramidal side effects (EPS) (e.g., akathisia, dystonia, Parkinsonism, acute dyskinesia, and tardive dyskinesia). In one variation, treatment with a combination therapy of the invention reduces or eliminates the number or extent of cognitive symptoms of schizophrenia (alleviates cognitive dysfunction) to a greater extent than therapies that do not comprise dosing with a first compound such as dimebon (e.g., when compared to the same or similar individuals who are on an antipsychotic individual/monotherapy or dual therapy where an atypical antipsychotic is administered in connection with a typical antipsychotic or where two or more atypical or typical antipsychotics are administered).

As used herein, unless clearly indicated otherwise, the term “an individual” intends a mammal, including but not limited to a human. The individual may be a human who has been diagnosed with or is suspected of having or is at risk of developing schizophrenia. The individual may be a human who exhibits one or more symptoms associated with schizophrenia. The individual may be a human who is genetically or otherwise predisposed to developing schizophrenia. In one variation, the individual may be a human who has been diagnosed with or is suspected of having or is at risk of developing schizophreniform disorder. In one variation, the individual may be a human who exhibits one or more symptoms associated with schizophreniform disorder. In one variation, the individual may be a human who is genetically or otherwise predisposed to developing schizophreniform disorder. In one variation, the individual may be a human who has been diagnosed with or is suspected of having or is at risk of developing schizoaffective disorder. In one variation, the individual may be a human who exhibits one or more symptoms associated with schizoaffective disorder. In one variation, the individual may be a human who is genetically or otherwise predisposed to developing schizoaffective disorder.

For use herein, unless clearly indicated otherwise, the combination therapy may be administered to the individual by any available dosage form. The first compound and second agent of a combination therapy may be administered in the same or different dosage forms and the invention includes these various dosage forms. In one variation, the first compound or the second agent or both the first compound and the second agent of a combination therapy is/are administered to the individual as a conventional immediate release dosage form. In one variation, the first compound or the second agent or both the first compound and the second agent of a combination therapy is/are administered to the individual as a sustained release form or part of a sustained release system, such as a system capable of sustaining the rate of delivery of the compound to an individual for a desired duration, which may be an extended duration such as a duration that is longer than the time required for a corresponding immediate-release dosage form to release the same amount (e.g., by weight or by moles) of compound, and can be hours or days. A desired duration may be at least the drug elimination half life of the administered compound and may be, e.g., at least about 6 hours or at least about 12 hours or at least about 24 hours or at least about 30 hours or at least about 48 hours or at least about 72 hours or at least about 96 hours or at least about 120 hours or at least about 144 or more hours, and can be at least about one week, at least about 2 weeks, at least about 3 weeks, at least about 4 weeks, at least about 8 weeks, or at least about 16 weeks or more.

The term “effective amount” intends such amount of a compound (e.g., a component of a combination therapy of the invention) or a combination therapy, which in combination with its parameters of efficacy and toxicity, as well as based on the knowledge of the practicing specialist should be effective in a given therapeutic form. As is understood in the art, an effective amount may be in one or more doses, i.e., a single dose or multiple doses may be required to achieve the desired treatment endpoint. In some embodiments, the effective amount of a compound or the combination therapy is an amount sufficient to reduce an activity of a 5HT2A receptor and a D2 receptor, such as a reduction of these activities by at least or about any of 10%, 20%, 30%, 40%, 50%, 60%, 70%, 80%, 90%, 95% or 100% as compared to the corresponding activity in the same subject prior to treatment or compared to the corresponding activity in other subjects not receiving the combination therapy. Standard methods can be used to measure the magnitude of this effect, such as in vitro assays with purified enzyme, cell-based assays, animal models, or human testing. An effective amount of a combination therapy includes an amount of the first compound and an amount of the second agent that, when administered sequentially, simultaneously, or continuously, produce a desired outcome.

In various embodiments, treatment with the combination therapy may result in an additive or even synergistic (e.g., greater than additive) result compared to administration of either the first compound or the second agent alone. In some embodiments, a lower amount of each of the first compound and the second agent is used as part of a combination therapy compared to the amount of each component generally used for individual (non-combination) therapy. Preferably, the same or greater therapeutic benefit is achieved using a combination therapy than using any of the individual compounds (combination components) alone. In some embodiments, the same or greater therapeutic benefit is achieved using a smaller amount (e.g., a lower dose or a less frequent dosing schedule) of a pharmaceutically active compound in a combination therapy than the amount generally used for individual therapy. Preferably, the use of a smaller amount of antipsychotic results in a reduction in the number, severity, frequency, or duration of one or more side-effects associated with that compound. Suitable doses of any of the compounds that are administered in conjunction with each other as part of the combination therapy may optionally be lowered due to the combined action (e.g., additive or synergistic effects) of the compounds.

The term “simultaneous administration,” as used herein, means that a first compound and a second agent in a combination therapy are administered with a time separation of no more than about 15 minutes, such as no more than about any of 10, 5, or 1 minutes. When the compounds are administered simultaneously, the first compound and second agent may be contained in the same composition (e.g., a composition comprising both a hydrogenated pyrido[4,3-b]indole such as dimebon and an antipsychotic such as the atypical antipsychotic risperidone and/or a typical antipsychotic such as perphenazine) or in separate compositions (e.g., a hydrogenated pyrido[4,3-b]indole such as dimebon is contained in one composition and an antipsychotic such as the atypical antipsychotic risperidone is contained in another composition).

As used herein, the term “sequential administration” means that the first compound and a second agent in a combination therapy are administered with a time separation of more than about 15 minutes, such as more than about any of 20, 30, 40, 50, 60 or more minutes. Either the first compound or the second agent may be administered first. The first compound and second agent for a sequential administration are contained in separate compositions, which may be contained in the same or different packages or kits.

A compound/component of the combination therapy may be formulated with suitable carriers for any available delivery route, whether in immediate or sustained release form, including oral, mucosal (e.g., nasal, sublingual, vaginal, buccal or rectal), parenteral (e.g., intramuscular, subcutaneous, or intravenous), topical or transdermal delivery. A compound may be formulated with suitable carriers to provide delivery forms, which may be but are not required to be sustained release forms, that include, but are not limited to: tablets, caplets, capsules (such as hard gelatin capsules and soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs. The first compound and second agent of a combination therapy may be formulated with suitable carriers for the same or different dosage routes and may be formulated for simultaneous administration via the same dosage route.

The first compound and second agent of a combination therapy can be used either separately or together in the preparation of a formulation, such as a pharmaceutical formulation, by combining the compound or compounds as an active ingredient with a pharmacologically acceptable carrier, which are known in the art. Depending on the therapeutic form of the system (e.g., transdermal patch vs. oral tablet), the carrier may be in various forms. In addition, pharmaceutical preparations may contain preservatives, solubilizers, stabilizers, re-wetting agents, emulgators, sweeteners, dyes, adjusters, salts for the adjustment of osmotic pressure, buffers, coating agents or antioxidants. Preparations containing an active ingredient may also contain other substances which have valuable therapeutic properties. Therapeutic forms may be represented by a usual standard dose and may be prepared by a known pharmaceutical method. Suitable formulations can be found, e.g., in Remington's Pharmaceutical Sciences, Mack Publishing Company, Philadelphia, Pa., 20^{th} ed. (2000), which is incorporated herein by reference.

The amount of a compound/component of the combination therapy in a delivery form may be any effective amount. In one variation, the combination therapy comprises the first compound (such as dimebon) in a dosage form in an amount from about 10 ng to about 1,500 mg or more. In one variation, the first compound (such as dimebon) in a dosage form comprises an amount from about 10 ng to about 1000 mg, from about 10 ng to about 500 mg, from about 10 ng to about 250 mg, from about 10 ng to about 100 mg, from about 10 ng to about 50 mg, from about 10 ng to about 25 mg, from about 10 ng to about 10 mg, from about 10 ng to about 5 mg, from about 10 ng to about 1 mg, from about 10 ng to about 500 μg, from about 10 ng to about 250 μg, from about 10 ng to about 100 μg, from about 10 ng to about 10 μg, from about 10 ng to about 5 μg, from about 10 ng to about 1 μg, from about 10 ng to about 500 ng, from about 10 ng to about 250 ng, from about 10 ng to about 100 ng, from about 10 ng to about 50 ng, or from about 10 ng to about 50 ng. In one variation, the first compound (such as dimebon) in a dosage form comprises an amount from about 10 ng to about 1000 ng, from about 100 ng to about 500 ng, from about 500 ng to about 1000 ng, from about 1 μg to about 100 μg, from about 10 μg to about 1000 μg, from about 100 μg to about 500 μg, from about 500 μg to about 1000 μg, from about 1 mg to about 100 mg, from about 10 mg to 100 mg, from about 50 mg to about 500 mg, from about 100 mg to 500 mg, from about 100 mg to about 1000 mg, or from about 500 mg to about 1500 mg. In one variation, the combination therapy comprises the second agent in a dosage form in an amount of from about 10 ng to about 1,500 mg or more. In one variation, the second agent is risperidone, and is administered in a dose of between 2 mg and 16 mg per day. In another variation, the second agent is risperidone, and is administered as an intramuscular depot formulation (e.g., Risperdal Consta) in a dose of between 25 mg to 50 mg every 2 weeks. In one variation, the combination therapy comprises dimebon as the first compound in a delivery form, such as a sustained release system, in an amount that is less than about 30 mg of dimebon. In one variation, the combination therapy comprises dimebon as the first compound in a delivery form, such as a single sustained release system capable of multi-day administration of dimebon, where the form comprises an amount of dimebon such that the daily dose of dimebon is less than about 30 mg.

A treatment regimen involving a dosage form of the first compound and/or a second agent of a combination therapy, whether immediate release or a sustained release system, may involve administering the first compound and/or the second agent to the individual in a dose of between about 0.1 and about 10 mg/kg of body weight, at least once a day and during the period of time required to achieve the therapeutic effect. In other variations, the daily dose (or other dosage frequency) of the first compound and/or the second agent is between about 0.1 and about 8 mg/kg; or between about 0.1 to about 6 mg/kg; or between about 0.1 and about 4 mg/kg; or between about 0.1 and about 2 mg/kg; or between about 0.1 and about 1 mg/kg; or between about 0.5 and about 10 mg/kg; or between about 1 and about 10 mg/kg; or between about 2 and about 10 mg/kg; or between about 4 to about 10 mg/kg; or between about 6 to about 10 mg/kg; or between about 8 to about 10 mg/kg; or between about 0.1 and about 5 mg/kg; or between about 0.1 and about 4 mg/kg; or between about 0.5 and about 5 mg/kg; or between about 1 and about 5 mg/kg; or between about 1 and about 4 mg/kg; or between about 2 and about 4 mg/kg; or between about 1 and about 3 mg/kg; or between about 1.5 and about 3 mg/kg; or between about 2 and about 3 mg/kg; or between about 0.01 and about 10 mg/kg; or between about 0.01 and 4 mg/kg; or between about 0.01 mg/kg and 2 mg/kg; or between about 0.05 and 10 mg/kg; or between about 0.05 and 8 mg/kg; or between about 0.05 and 4 mg/kg; or between about 0.05 and 4 mg/kg; or between about 0.05 and about 3 mg/kg; or between about 10 kg to about 50 kg; or between about 10 to about 100 mg/kg or between about 10 to about 250 mg/kg; or between about 50 to about 100 mg/kg or between about 50 and 200 mg/kg; or between about 100 and about 200 mg/kg or between about 200 and about 500 mg/kg; or a dosage over about 100 mg/kg; or a dosage over about 500 mg/kg. In some embodiments, a daily dosage of dimebon as the first compound of a combination therapy is administered, such as a daily dosage of dimebon is less than about 0.1 mg/kg, which may include but is not limited to, a daily dosage of about 0.05 mg/kg. In one variation, the daily dose (by weight) of the first compound (such as dimebon) is about 10 times the daily dose (by weight) of the second agent. For instance, in one variation, the combination therapy involves administering dimebon in a daily dose of about 60 mg and risperidone in a daily dose of about 6 mg.

The combination therapy may be administered to an individual in accordance with an effective dosing regimen for a desired period of time or duration, such as at least about one month, at least about 2 months, at least about 3 months, at least about 6 months, or at least about 12 months or longer. In one variation, the combination therapy is administered on a daily or intermittent schedule for the duration of the individual's life.

The dosing frequency of the first compound and/or the second agent in a combination therapy can be about a once weekly dosing. The dosing frequency of the first compound and/or the second agent in a combination therapy can be about a once daily dosing. The dosing frequency of the first compound and/or the second agent in a combination therapy can be more than about once weekly dosing. The dosing frequency of the first compound and/or the second agent in a combination therapy can be less than three times a day dosing. The dosing frequency of the first compound and/or the second agent in a combination therapy can be less than about three times a day dosing. The dosing frequency of the first compound and/or the second agent in a combination therapy can be about three times a week dosing. The dosing frequency of the first compound and/or the second agent in a combination therapy can be about a four times a week dosing. The dosing frequency of the first compound and/or the second agent in a combination therapy can be about a two times a week dosing. The dosing frequency of the first compound and/or the second agent in a combination therapy can be more than about once weekly dosing but less than about daily dosing. The dosing frequency of the first compound and/or the second agent in a combination therapy can be about a once monthly dosing. The dosing frequency of the first compound and/or the second agent in a combination therapy can be about a twice weekly dosing. The dosing frequency of the first compound and/or the second agent in a combination therapy can be more than about once monthly dosing but less than about once weekly dosing. The dosing frequency of the first compound and/or the second agent in a combination therapy can be intermittent (e.g., once daily dosing for 7 days followed by no doses for 7 days, repeated for any 14 day time period, such as about 2 months, about 4 months, about 6 months or more). The dosing frequency of the first compound and/or the second agent in a combination therapy can be continuous (e.g., once weekly dosing for continuous weeks). Any of the dosing frequencies can employ any of the compounds described herein together with any of the dosages described herein, for example, the dosing frequency of the first compound in a combination therapy can be a once daily dosage of less than 0.1 mg/kg or less than about 0.05 mg/kg of dimebon. In one variation, the dosing of the first compound is three times daily and the dosing of the second agent is once daily. In a particular variation the combination therapy involves administration of dimebon three times daily (e.g., about 20 mg administered 3 times daily) and administration of risperidone once daily (e.g., about 6 mg administered once daily).

### Methods for Treating Schizophrenia

The hydrogenated pyrido[4,3-b]indoles and antipsychotics described herein may be used in a combination therapy to treat and/or prevent and/or delay the onset and/or the development of schizophrenia, including its positive, negative, and/or cognitive symptoms. As illustrated in Example 1, the representative hydrogenated pyrido[4,3-b]indole dimebon is capable of reducing the blocking effect of MK-801 on NMDA-induced currents in cultured rat hippocampus neurons. Exemplary methods for determining the ability of hydrogenated pyrido[4,3-b]indoles to treat and/or prevent and/or delay the onset and/or the development of schizophrenia are described in Examples 2 and 3. An ongoing human study involving combination therapy is described in Example 4.

It was surprisingly found that dimebon, although an NMDA receptor blocker, may also be capable of reducing the blocking activity of MK-801 on NMDA receptors. Since it was found that phencyclidine and MK-801 act in accordance with the same mechanism, by competing for the same intrachannel segment of the NMDA receptor it should be expected that the first compounds described herein, such as dimebon, will weaken the blocking effect of phencyclidine on the NMDA receptor in exactly the same way. Since the psychotomimetic properties of phencyclidine are due to its ability to stably bind to a specific segment within the NMDA receptor ion channel and to block ion currents passing through its ion channel, then the attenuation of this blocking effect by compounds described herein, such as those of Formula (1), (2), (A) or (B) should lead to a decrease of the psychotomimetic properties of phencyclidine.

Atypical and typical antipsychotics have found use in the treatment of schizophrenia. For example, risperidone is an atypical antipsychotic that has been approved in the United States for the treatment of schizophrenia. Risperidone is available as a tablet in strengths ranging from 0.25 mg to 4 mg, as an oral solution, such as 1 mg/mL and as disintegrating tables, such as in strengths ranging from 0.5 to 4 mg. However, use of atypical antipsychotics is not without side effects, such as the potential to cause tardive dyskinesia and extrapyramidal symptoms (ESP), which are characterized by involuntary movements, as well as weight gain, metabolic syndrome, prolonged QT interval, hypotension, sedation, and neuroleptic malignant syndrome. Certain atypical antipsychotics may also have limited use in particular patient populations.

Combination therapies that include a first compound and a second agent, where the first compound is a hydrogenated pyrido[4,3-b]indole, particularly the compound dimebon, and the second agent is an antipsychotic, may have enhanced activity for treating, preventing and/or delaying the onset and/or development of schizophrenia. In particular, combination therapies of the invention include a hydrogenated pyrido[4,3-b]indole or a pharmaceutically acceptable salt thereof in conjunction with an antipsychotic useful for treating, preventing and/or delaying the onset and/or development of schizophrenia. Methods that use such combination therapies may result in an additive or even synergistic (e.g., greater than additive) result compared to administration of either compound of the combination therapy alone.

In one variation, a combination therapy comprising a first compound and a second agent requires lower doses of the individual compounds than would be necessary if the individual compounds were given alone. This decreased dosage may reduce side-effects associated with the therapies and result in greater patient compliance, which is highly desirable for the schizophrenic patient population. Thus, in some embodiments, a lower amount of each pharmaceutically active compound is used as part of a combination therapy compared to the amount generally used for individual therapy. In some embodiments, the same or greater therapeutic benefit is achieved using a smaller amount (e.g., a lower dose or a less frequent dosing schedule) of a pharmaceutically active compound in a combination therapy than the amount generally used for individual therapy. Preferably, the use of a small amount of pharmaceutically active compound results in a reduction in the number, severity, frequency or duration of one or more side-effects associated with the compound.

Thus, the present invention provides a variety of methods using combination therapy, such as those described in the “Brief Summary of the Invention” and elsewhere in this disclosure. For example, in one embodiment, the present invention provides a method of treating schizophrenia in a patient in need thereof comprising administering to the individual an effective amount of a combination therapy comprising a hydrogenated pyrido[4,3-b]indole or pharmaceutically acceptable salt thereof (such as dimebon) and an antipsychotic. In one embodiment, the present invention provides a method of delaying the onset and/or development of schizophrenia in an individual who is considered at risk for developing schizophrenia (e.g., an individual whose one or more family members have had schizophrenia or an individual who has been diagnosed as having a genetic mutation associated with schizophrenia or an individual who exhibits behavior consistent with the onset of schizophrenia) comprising administering to the individual an effective amount of a combination therapy comprising a hydrogenated pyrido[4,3-b]indole or pharmaceutically acceptable salt thereof (such as dimebon) and an antipsychotic. In one embodiment, the present invention provides a method of delaying the onset and/or development of schizophrenia in an individual who is genetically predisposed to developing schizophrenia comprising administering to the individual an effective amount of a combination therapy comprising a hydrogenated pyrido[4,3-b]indole or pharmaceutically acceptable salt thereof (such as dimebon) and an antipsychotic. In one embodiment, the present invention provides a method of delaying the onset and/or development schizophrenia in an individual having a mutated or abnormal gene associated with schizophrenia (such as the NRG1 or DTNBP1 gene) but who has not been diagnosed with schizophrenia comprising administering to the individual an effective amount of a combination therapy comprising a hydrogenated pyrido[4,3-b]indole or pharmaceutically acceptable salt thereof (such as dimebon) and an antipsychotic. In one embodiment, the present invention provides a method of preventing the onset and/or development of schizophrenia in an individual who is genetically predisposed to developing schizophrenia or who has a mutated or abnormal gene associated with schizophrenia but who has not been diagnosed with schizophrenia comprising administering to the individual an effective amount of a combination therapy comprising a hydrogenated pyrido[4,3-b]indole or pharmaceutically acceptable salt thereof (such as dimebon) and an antipsychotic. In one embodiment, the present invention provides a method of preventing the onset and/or development of schizophrenia in an individual who is not identified as genetically predisposed to developing schizophrenia comprising administering to the individual an effective amount of a combination therapy comprising a hydrogenated pyrido[4,3-b]indole or pharmaceutically acceptable salt thereof (such as dimebon) and an antipsychotic. In one embodiment, the present invention provides a method of decreasing the intensity or severity of the symptoms of schizophrenia in an individual who is diagnosed with schizophrenia comprising administering to the individual an effective amount of a combination therapy comprising a hydrogenated pyrido[4,3-b]indole or pharmaceutically acceptable salt thereof (such as dimebon) and an antipsychotic. In one embodiment, the present invention provides a method of enhancing the quality of life of an individual diagnosed with schizophrenia comprising administering to the individual an effective amount of a combination therapy comprising a hydrogenated pyrido[4,3-b]indole or pharmaceutically acceptable salt thereof (such as dimebon) and an antipsychotic. In one variation, the method comprises the manufacture of a combination therapy medicament for use in any of the described methods, e.g., treating and/or preventing and/or delaying the onset or development of schizophrenia. In one variation, the methods of the invention employ a combination therapy whereby the antipsychotic is other than an atypical antipsychotic. In one variation, the antipsychotic is an atypical antipsychotic. In one variation, the atypical antipsychotic is selected from the group consisting of risperidone, clozapine, N-desmethylclozapine, olanzapine, quetiapine, perospirone, ziprasidone, olanzapine/fluoxetine (marketed as Symbyax™), aripiprazole, paliperidone, sertindole, zotepine, amisulpride, bifeprunox, asenapine, melperone, abaperidone, blonanserin, iloperidone, lurasidone, ocaperidone, QF-2400B, SB-773812, ITI-007, and YKP-1358. In one variation, the atypical antipsychotic is risperidone. In one variation, the antipsychotic is a typical antipsychotic. In one variation, the typical antipsychotic is selected from the group consisting of chlorpromazine, trifluoroperazine hydrochloride, fluphenazine HCl or fluphenazine decanoate, haloperidol, molindone, thiothixene, thioridazine, trifluoperazine, loxapine, perphenazine, prochlorperazine, pimozide, and zuclopenthixol. In one variation, the typical antipsychotic is perphenazine. In one variation, the antipsychotic is a combination of an atypical antipsychotic and a typical antipsychotic (in which case the second agent could contain at least two different compounds). In one variation, the antipsychotic is a combination of an atypical antipsychotic selected from the group consisting of risperidone, clozapine, N-desmethylclozapine, olanzapine, quetiapine, perospirone, ziprasidone, olanzapine/fluoxetine (marketed as Symbyax™), aripiprazole, paliperidone, sertindole, zotepine, amisulpride, bifeprunox, asenapine, melperone, abaperidone, blonanserin, iloperidone, lurasidone, ocaperidone, QF-2400B, SB-773812, ITI-007, and YKP-1358, and a typical antipsychotic selected from the group consisting of chlorpromazine, trifluoroperazine hydrochloride, fluphenazine HCl or fluphenazine decanoate, haloperidol, molindone, thiothixene, thioridazine, trifluoperazine, loxapine, perphenazine, prochlorperazine, pimozide, and zuclopenthixol. In one variation, the antipsychotic is a combination of the atypical antipsychotic risperidone and the typical antipsychotic perphenazine.

Thus, the invention provides methods of treating schizophrenia comprising administering a hydrogenated pyrido[4,3-b]indole or pharmaceutically acceptable salt thereof (such as dimebon) and an antipsychotic (such as risperidone and/or perphenazine) wherein the individual has (or is suspected of having) schizophrenia. Methods of administering an antipsychotic such as risperidone and/or perphenazine are known in the art. Reducing the dosage of an antipsychotic (which reduces the dependence on administration of these drugs and in effect delays administration of these drugs) can be assessed by, for example, comparing to known and/or established averages of dosage (in terms of amount and/or intervals) generally given over time which are known in the art.

In another aspect, the invention provides methods for enhancing treatment of schizophrenia with an antipsychotic comprising administering an effective amount of a hydrogenated pyrido[4,3-b]indole or pharmaceutically acceptable salt thereof (such as dimebon) in conjunction with an antipsychotic. Enhanced treatment can be assessed by evaluating known parameters and/or indicators (such as the number and/or severity of symptoms and/or clinical and/or psychometric and/or neurocognitive and/or biological markers or assessments) in an individual who is given a combination therapy as compared to the same parameters and/or indicators in the same or similar individuals who are given antipsychotic monotherapy or who are not on a combination therapy comprising a first compound as described herein.

Hydrogenated pyrido[4,3-b]indole Compounds for Use in the Methods, Formulations, Kits and Inventions Disclosed Herein

When reference to organic residues or moieties having a specific number of carbons is made, unless clearly stated otherwise, it intends all geometric and other isomers thereof. For example, “butyl” includes n-butyl, sec-butyl, isobutyl and t-butyl; “propyl” includes n-propyl and isopropyl.

The term “alkyl” intends and includes linear, branched or cyclic hydrocarbon structures and combinations thereof. Preferred alkyl groups are those having 20 carbon atoms (C20) or fewer. More preferred alkyl groups are those having fewer than 15 or fewer than 10 or fewer than 8 carbon atoms.

The term “lower alkyl” refers to alkyl groups of from 1 to 5 carbon atoms. Examples of lower alkyl groups include methyl, ethyl, propyl, isopropyl, butyl, s- and t-butyl and the like. Lower alkyl is a subset of alkyl.

The term “aryl” or (“Ar”) refers to an unsaturated aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxain-3(4H)-one-7-yl), and the like. Preferred aryls includes phenyl and naphthyl.

The term “heteroaryl” refers to an aromatic carbocyclic group of from 2 to 10 carbon atoms and 1 to 4 heteroatoms selected from oxygen, nitrogen and sulfur within the ring. Such heteroaryl groups can have a single ring (e.g., pyridyl or furyl) or multiple condensed rings (e.g., indolizinyl or benzothienyl). Examples of heteroaryl residues include, e.g., imidazolyl, pyridinyl, indolyl, thiopheneyl, thiazolyl, furanyl, benzimidazolyl, quinolinyl, isoquinolinyl, pyrimidinyl, pyrazinyl, tetrazolyl and pyrazolyl.

The term “aralkyl” refers to a residue in which an aryl moiety is attached to the parent structure via an alkyl residue. Examples are benzyl, phenethyl and the like.

The term “heteroaralkyl” refers to a residue in which a heteroaryl moiety is attached to the parent structure via an alkyl residue. Examples include furanylmethyl, pyridinylmethyl, pyrimidinylethyl and the like.

The term “substituted heteroaralkyl” refers to heteroaryl groups which are substituted with from 1 to 3 substituents, such as residues selected from the group consisting of hydroxy, alkyl, alkoxy, alkenyl, alkynyl, amino, aryl, carboxyl, halo, nitro and amino.

The term “halo” or “halogen” refers to fluoro, chloro, bromo and iodo.

Hydrogenated pyrido[4,3-b]indoles or pharmaceutically acceptable salts thereof, such as an acid or base salt thereof, are the first compound of a combination therapy containing a hydrogenated pyrido[4,3-b]indole and an antipsychotic. A hydrogenated pyrido[4,3-b]indole can be a tetrahydro pyrido[4,3-b]indole or pharmaceutically acceptable salt thereof. The hydrogenated pyrido[4,3-b]indole can also be a hexahydro pyrido[4,3-b]indole or pharmaceutically acceptable salt thereof. The hydrogenated pyrido[4,3-b]indole compounds can be substituted with 1 to 3 substituents, although unsubstituted hydrogenated pyrido[4,3-b]indole compounds or hydrogenated pyrido[4,3-b]indole compounds with more than 3 substituents are also contemplated. Suitable substituents include but are not limited to alkyl, lower alkyl, aralkyl, heteroaralkyl, substituted heteroaralkyl, and halo.

Particular hydrogenated pyrido[4,3-b]indoles are exemplified by the Formulae A and B:

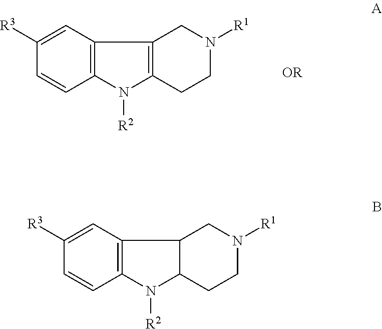

where R¹ is selected from the group consisting of alkyl, lower alkyl and aralkyl, R² is selected from the group consisting of hydrogen, aralkyl and substituted heteroaralkyl; and R³ is selected from the group consisting of hydrogen, alkyl, lower alkyl and halo.

In one variation, R¹ is alkyl, such as an alkyl selected from the group consisting of C₁-C₁₅alkyl, C₁₀-C₁₅alkyl, C₁-C₁₀alkyl, C₂-C₁₅alkyl, C₂-C₁₀alkyl, C₂-C₈alkyl, C₄-C₈alkyl, C₆-C₈alkyl, C₆-C₁₅alkyl, C₁₅-C₂₀alkyl; C₁-C₈alkyl and C₁-C₆alkyl. In one variation, R¹ is aralkyl. In one variation, R¹ is lower alkyl, such as a lower alkyl selected from the group consisting of C₁-C₂alkyl, C₁-C₄alkyl, C₂-C₄ alkyl, C₁-C₅ alkyl, C₁-C₃alkyl, and C₂-C₅alkyl.

In one variation, R¹ is a straight chain alkyl group. In one variation, R¹ is a branched alkyl group. In one variation, R¹ is a cyclic alkyl group.

In one variation, R¹ is methyl. In one variation, R¹ is ethyl. In one variation, R¹ is methyl or ethyl. In one variation, R¹ is methyl or an aralkyl group such as benzyl. In one variation, R¹ is ethyl or an aralkyl group such as benzyl.

In one variation, R¹ is an aralkyl group. In one variation, R¹ is an aralkyl group where any one of the alkyl or lower alkyl substituents listed in the preceding paragraphs is further substituted with an aryl group (e.g., Ar—C₁-C₆alkyl, Ar—C₁-C₃alkyl or Ar—C₁-C₁₅alkyl). In one variation, R¹ is an aralkyl group where any one of the alkyl or lower alkyl substituents listed in the preceding paragraphs is substituted with a single ring aryl residue. In one variation, R¹ is an aralkyl group where any one of the alkyl or lower alkyl substituents listed in the preceding paragraphs is further substituted with a phenyl group (e.g., Ph-C₁-C₆Alkyl or Ph-C₁-C₃Alkyl, Ph-C₁-C₁₅alkyl). In one variation, R¹ is benzyl.

All of the variations for R¹ are intended and hereby clearly described to be combined with any of the variations stated below for R² and R³ the same as if each and every combination of R¹, R² and R³ were specifically and individually listed.

In one variation, R² is H. In one variation, R² is an aralkyl group. In one variation, R² is a substituted heteroaralkyl group. In one variation, R² is hydrogen or an aralkyl group. In one variation, R² is hydrogen or a substituted heteroaralkyl group. In one variation, R² is an aralkyl group or a substituted heteroaralkyl group. In one variation, R² is selected from the group consisting of hydrogen, an aralkyl group and a substituted heteroaralkyl group.

In one variation, R² is an aralkyl group where R² can be any one of the aralkyl groups noted for R¹ above, the same as if each and every aralkyl variation listed for R¹ is separately and individually listed for R².

In one variation, R² is a substituted heteroaralkyl group, where the alkyl moiety of the heteroaralkyl can be any alkyl or lower alkyl group, such as those listed above for R¹. In one variation, R² is a substituted heteroaralkyl where the heteroaryl group is substituted with 1 to 3 C₁-C₃ alkyl substituents (e.g., 6-methyl-3-pyridylethyl). In one variation, R² is a substituted heteroaralkyl group wherein the heteroaryl group is substituted with 1 to 3 methyl groups. In one variation, R² is a substituted heteroaralkyl group wherein the heteroaryl group is substituted with one lower alkyl substituent. In one variation, R² is a substituted heteroaralkyl group wherein the heteroaryl group is substituted with one C₁-C₃ alkyl substituent. In one variation, R² is a substituted heteroaralkyl group wherein the heteroaryl group is substituted with one or two methyl groups. In one variation, R² is a substituted heteroaralkyl group wherein the heteroaryl group is substituted with one methyl group.

In other variations, R² is any one of the substituted heteroaralkyl groups in the immediately preceding paragraph where the heteroaryl moiety of the heteroaralkyl group is a single ring heteroaryl group. In other variations, R² is any one of the substituted heteroaralkyl groups in the immediately preceding paragraph where the heteroaryl moiety of the heteroaralkyl group is a multiple condensed ring heteroaryl group. In other variations, R² is any one of the substituted heteroaralkyl groups in the immediately preceding paragraph where the heteroaralkyl moiety is a pyridyl group (Py).

In one variation, R² is 6-CH₃-3-Py-(CH₂)₂—.

In one variation, R³ is hydrogen. In other variations, R³ is any one of the alkyl groups noted for R¹ above, the same as if each and every alkyl variation listed for R¹ is separately and individually listed for R³. In another variation, R³ is a halo group. In one variation, R³ is hydrogen or an alkyl group. In one variation, R³ is a halo or alkyl group. In one variation, R³ is hydrogen or a halo group. In one variation, R³ is selected from the group consisting of hydrogen, alkyl and halo. In one variation, R³ is Br. In one variation, R³ is I. In one variation, R³ is F. In one variation, R³ is Cl.

In a particular variation, the hydrogenated pyrido[4,3-b]indole is 2,8-dimethyl-5-(2-(6-methyl-3-pyridyl)ethyl)-2,3,4,5-tetrahydro-1H-pyrido[4,3-b]indole or a pharmaceutically acceptable salt thereof.

The hydrogenated pyrido[4,3-b]indoles can be in the form of pharmaceutically acceptable salts thereof, which are readily known to those of skill in the art. The pharmaceutically acceptable salts include pharmaceutically acceptable acid salts. Examples of particular pharmaceutically acceptable salts include hydrochloride salts or dihydrochloride salts. In a particular variation, the hydrogenated pyrido[4,3-b]indole is a pharmaceutically acceptable salt of 2,8-dimethyl-5-(2-(6-methyl-3-pyridyl)ethyl)-2,3,4,5-tetrahydro-1H-pyrido[4,3-b]indole, such as 2,8-dimethyl-5-(2-(6-methyl-3-pyridyl)ethyl)-2,3,4,5-tetrahydro-1H-pyrido[4,3-b]indole dihydrochloride (dimebon).

Particular hydrogenated pyrido-([4,3-b])indoles can also be described by the Formula (1) or by the Formula (2):

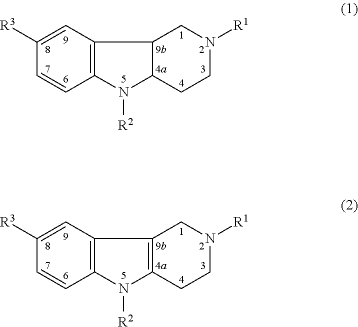

For compounds of a general Formula (1) or (2), R¹ represents —CH₃, CH₃CH₂—, or PhCH₂-(benzyl); R² is —H, PhCH₂—, or 6-CH₃-3-Py-(CH₂)₂—; R³ is —H, —CH₃, or —Br, in any combination of the above substituents. All possible combinations of the substituents of Formulae (1) and (2) are contemplated as specific and individual compounds the same as if each single and individual compound were listed by chemical name. Also contemplated are the compounds of Formula (1) or (2), with any deletion of one or more possible moieties from the substituent groups listed above: e.g., where R¹ represents —CH₃; R² is —H, PhCH₂—, or 6-CH₃-3-Py-(CH₂)₂—; and R³ is —H, —CH₃, or —Br, or where R¹ represents —CH₃; R² is 6-CH₃-3-Py-(CH₂)₂—; and R³ represents —H, —CH₃, or —Br.

The above and any compound herein may be in a form of salts with pharmaceutically acceptable acids and in a form of quaternized derivatives.

The compound may be Formula (1), where R¹ is —CH₃, R² is —H, and R³ is —CH₃. In one variation, the compound is of the Formula (1), provided that the substituents are not where R¹ is —CH₃, R²—H, and R³ is —CH₃. The compound may be Formula (2), where R¹ is represented by —CH₃, CH₃CH₂—, or PhCH₂—; R² is —H, PhCH₂—, or 6-CH₃-3-Py-(CH₂)₂—; R³ is —H, —CH₃, or —Br. The compound may be Formula (2), where R¹ is CH₃CH₂— or PhCH₂—, R² is —H, and R³ is —H; or a compound, where R¹ is —CH₃, R² is PhCH₂—, R³ is —CH₃; or a compound, where R¹ is —CH₃, R² is 6-CH₃-3-Py-(CH₂)₂—, and R³ is —CH₃; or a compound, where R¹ is —CH₃, R² is —H, R³ is —H or —CH₃; or a compound, where R¹ is —CH₃, R² is —H, R³ is —Br.

Compounds known from literature which can be used in the methods disclosed herein include the following specific compounds:
1. cis(±) 2,8-dimethyl-2,3,4,4a,5,9b-hexahydro-1H-pyrido[4,3-b]indole and its dihydrochloride;
2. 2-ethyl-2,3,4,5-tetrahydro-1H-pyrido[4,3-b]indole;
3. 2-benzyl-2,3,4,5-tetrahydro-1H-pyrido[4,3-b]indole;
4. 2,8-dimethyl-5-benzyl-2,3,4,5-tetrahydro-1H-pyrido[4,3-b]indole and its dihydrochloride;
5. 2-methyl-5-(2-methyl-3-pyridyl)ethyl-2,3,4,5-tetrahydro-1H-pyrido[4,3-b]indole and its sesquisulfate;
6. 2,8-dimethyl-5-(2-(6-methyl-3-pyridyl)ethyl)-2,3,4,5-tetrahydro-1H-pyrido[4,3-b]indole and its dihydrochloride (dimebon);
7. 2-methyl-2,3,4,5-tetrahydro-1H-pyrido[4,3-b]indole;
8. 2,8-dimethyl-2,3,4,5-tetrahydro-1H-pyrido[4,3-b]indole and its methyl iodide;
9. 2-methyl-8-bromo-2,3,4,5-tetrahydro-1H-pyrido[4,3-b]indole and its hydrochloride.

In one variation, the compound is of the Formula A or B and R¹ is selected from a lower alkyl or benzyl; R² is selected from a hydrogen, benzyl or 6-CH₃-3-Py-(CH₂)₂— and R³ is selected from hydrogen, lower alkyl or halo, or any pharmaceutically acceptable salt thereof. In another variation, R¹ is selected from —CH₃, CH₃CH₂—, or benzyl; R² is selected from —H, benzyl, or 6-CH₃-3-Py-(CH₂)₂—; and R³ is selected from —H, —CH₃ or —Br, or any pharmaceutically acceptable salt thereof. In another variation the compound is selected from the group consisting of: cis(±) 2,8-dimethyl-2,3,4,4a,5,9b-hexahydro-1H-pyrido[4,3-b]indole as a racemic mixture or in the substantially pure (+) or substantially pure (−) form; 2-ethyl-2,3,4,5-tetrahydro-1H-pyrido[4,3-b]indole; 2-benzyl-2,3,4,5-tetrahydro-1H-pyrido[4,3-b]indole; 2,8-dimethyl-5-benzyl-2,3,4,5-tetrahydro-1H-pyrido[4,3-b]indole; 2-methyl-5-(2-methyl-3-pyridyl)ethyl-2,3,4,5-tetrahydro-1H-pyrido[4,3-b]indole; 2,8-dimethyl-5-(2-(6-methyl-3-pyridyl)ethyl)-2,3,4,5-tetrahydro-1H-pyrido[4,3-b]indole; 2-methyl-2,3,4,5-tetrahydro-1H-pyrido[4,3-b]indole; 2,8-dimethyl-2,3,4,5-tetrahydro-1H-pyrido[4,3-b]indole; or 2-methyl-8-bromo-2,3,4,5-tetrahydro-1H-pyrido[4,3-b]indole or any pharmaceutically acceptable salt of any of the foregoing. In one variation, the compound is of the Formula A or B wherein R¹ is —CH₃, R² is —H and R³ is —CH₃ or any pharmaceutically acceptable salt thereof. The compound may be of the Formula A or B where R¹ is CH₃CH₂— or benzyl, R² is —H, and R³ is —CH₃ or any pharmaceutically acceptable salt thereof. The compound may be of the Formula A or B where R¹ is —CH₃, R² is benzyl, and R³ is —CH₃ or any pharmaceutically acceptable salt thereof. The compound may be of the Formula A or B where R¹ is —CH₃, R² is 6-CH₃-3-Py-(CH₂)₂—, and R³ is —H or any pharmaceutically acceptable salt thereof. The compound may be of the Formula A or B where R² is 6-CH₃-3-Py-(CH₂)₂— or any pharmaceutically acceptable salt thereof. The compound may be of the Formula A or B where R¹ is —CH₃, R² is —H, and R³ is —H or —CH₃ or any pharmaceutically acceptable salt, thereof. The compound may be of the Formula A or B where R¹ is —CH₃, R² is —H, and R³ is —Br, or any pharmaceutically acceptable salt thereof. The compound may be of the Formula A or B where R¹ is selected from a lower alkyl or aralkyl, R² is selected from a hydrogen, aralkyl or substituted heteroaralkyl and R³ is selected from hydrogen, lower alkyl or halo.

The compound for use in the compositions, kits and methods may be 2,8-dimethyl-5-(2-(6-methyl-3-pyridyl)ethyl-2,3,4,5-tetrahydro-1H-pyrido[4,3-b]indole or any pharmaceutically acceptable salt thereof, such as an acid salt, a hydrochloride salt or a dihydrochloride salt thereof.

Any of the compounds disclosed herein having two stereocenters in the pyrido[4,3-b]indole ring structure (e.g., carbons 4a and 9b of compound (1)) includes compounds whose stereocenters are in a cis or a trans form. A composition may comprise such a compound in substantially pure form, such as a composition of substantially pure S,S or R,R or S,R or R,S compound. A composition of substantially pure compound means that the composition contains no more than 15% or no more than 10% or no more than 5% or no more than 3% or no more than 1% impurity of the compound in a different stereochemical form. For instance, a composition of substantially pure S,S compound means that the composition contains no more than 15% or no more than 10% or no more than 5% or no more than 3% or no more than 1% of the R,R or S,R or R,S form of the compound. A composition may contain the compound as mixtures of such stereoisomers, where the mixture may be enanteomers (e.g., S,S and R,R) or diastereomers (e.g., S,S and R,S or S,R) in equal or unequal amounts. A composition may contain the compound as a mixture of 2 or 3 or 4 such stereoisomers in any ratio of stereoisomers. Compounds disclosed herein having stereocenters other than in the pyrido[4,3-b]indole ring structure intends all stereochemical variations of such compounds, including but not limited to enantiomers and diastereomers in any ratio, and includes racemic and enantioenriched and other possible mixtures. Unless stereochemistry is explicitly indicated in a structure, the structure is intended to embrace all possible stereoisomers of the compound depicted.

Synthesis and studies on neuroleptic properties for cis(±) 2,8-dimethyl-2,3,4,4a,5,9b-hexahydro-1H-pyrido[4,3-b]indole and its dihydrochloride are reported, for instance, in the following publication: Yakhontov, L. N., Glushkov, R. G., Synthetic therapeutic drugs. A. G. Natradze, the editor, Moscow Medicina, 1983, p. 234-237. Synthesis of compounds 2, 8, and 9 noted above as known from the literature, and data on their properties as serotonin antagonists are reported in, for instance, in C. J. Cattanach, A. Cohen & B. H. Brown in J. Chem. Soc. (Ser. C) 1968, p. 1235-1243. Synthesis of the compound 3 noted above as known from the literature is reported, for instance, in the article N. P. Buu-Hoi, O. Roussel, P. Jacquignon, J. Chem. Soc., 1964, N 2, p. 708-711. N. F. Kucherova and N. K. Kochetkov (General chemistry (russ.), 1956, v. 26, p. 3149-3154) describe the synthesis of the compound 4 noted above as known from the literature. Synthesis of compounds 5 and 6 noted above as known from the literature is described in the article by A. N. Kost, M. A. Yurovskaya, T. V. Mel'nikova, in Chemistry of heterocyclic compounds, 1973, N 2, p. 207-212. The synthesis of the compound 7 noted above as known from the literature is described by U, Horlein in Chem. Ber., 1954, Bd. 87, hft 4, 463-p. 472. M. Yurovskaya and I. L. Rodionov in Chemistry of heterocyclic compounds (1981, N 8, p. 1072-1078) describe the synthesis of methyl iodide of the compound 8 above.

The first compound and the second agents of a combination therapy may be combined with a pharmaceutically acceptable carrier, and pharmaceutical compositions comprising the combination therapy are intended.

The invention also embraces combination therapy unit dosage forms, where the first compound and the second agent of a combination therapy are present in a unit dosage form. As used herein, the term “unit dosage form” refers to a combination therapy formulation that contains a predetermined dose of a first compound (such as dimebon) and a predetermined dose of a second agent (such as risperidone). The first compound and the second agents of the combination therapy unit dosage form are present in amounts effective to treat schizophrenia.

The invention further provides kits comprising a combination therapy as described herein. The kits may contain the first compound and the second agents of the combination therapy as a unit dosage form (e.g., the dosage form contains both dimebon and an antipsychotic such as risperidone and/or perphenazine) or as discrete dosage forms (e.g., dimebon is contained in one dosage form and the antipsychotic such as risperidone and/or perphenazine is contained in another dosage form). The kits will also contain instructions for use. In one variation, the kits comprise (a) dimebon, (b) an antipsychotic; and (c) instructions for use of in the treatment, prevention, slowing the progression or delaying the onset and/or development of schizophrenia. In one variation, the antipsychotic is an atypical antipsychotic. In one variation, the atypical antipsychotic is selected from the group consisting of risperidone, clozapine, N-desmethylclozapine, olanzapine, quetiapine, perospirone, ziprasidone, olanzapine/fluoxetine (marketed as Symbyax™), aripiprazole, paliperidone, sertindole, zotepine, amisulpride, bifeprunox, asenapine, melperone, abaperidone, blonanserin, iloperidone, lurasidone, ocaperidone, QF-2400B, SB-773812, ITI-007, and YKP-1358. In one variation, the atypical antipsychotic is risperidone. In one variation, the antipsychotic is a typical antipsychotic. In one variation, the typical antipsychotic is selected from the group consisting of chlorpromazine, trifluoroperazine hydrochloride, fluphenazine HCl or fluphenazine decanoate, haloperidol, molindone, thiothixene, thioridazine, trifluoperazine, loxapine, perphenazine, prochlorperazine, pimozide, and zuclopenthixol. In one variation, the typical antipsychotic is perphenazine. In one variation, the kit employs dimebon and risperidone. In one variation, the kit employs dimebon and perphenazine. In one variation, the antipsychotic is a combination of an atypical antipsychotic and a typical antipsychotic (in which case the second agent could contain at least two different compounds). In one variation, the antipsychotic is a combination of an atypical antipsychotic selected from the group consisting of risperidone, clozapine, N-desmethylclozapine, olanzapine, quetiapine, perospirone, ziprasidone, olanzapine/fluoxetine (marketed as , Symbyax™), aripiprazole, paliperidone, sertindole, zotepine, amisulpride, bifeprunox, asenapine, melperone, abaperidone, blonanserin, iloperidone, lurasidone, ocaperidone, QF-2400B, SB-773812, ITI-007, and YKP-1358, and a typical antipsychotic selected from the group consisting of chlorpromazine, trifluoroperazine hydrochloride, fluphenazine HCl or fluphenazine decanoate, haloperidol, molindone, thiothixene, thioridazine, trifluoperazine, loxapine, perphenazine, prochlorperazine, pimozide, and zuclopenthixol. In one variation, the kit employs dimebon and a combination of the atypical antipsychotic risperidone and the typical antipsychotic perphenazine. The kits may be used for any one or more of the uses described herein, and, accordingly, may contain instructions for any one or more of the stated uses (e.g., treating and/or preventing and/or delaying the onset and/or the development of schizophrenia).

Kits generally comprise suitable packaging. The kits may comprise one or more containers comprising any compound or combination therapy described herein. Each component (if there is more than one component) can be packaged in separate containers or some components can be combined in one container where cross-reactivity and shelf life permit.

The kits may optionally include a set of instructions, generally written instructions, although electronic storage media (e.g., magnetic diskette or optical disk) containing instructions are also acceptable, relating to the use of component(s) of the methods of the present invention (e.g., treating, preventing and/or delaying the onset and/or the development of schizophrenia. The instructions included with the kit generally include information as to the components and their administration to an individual.

The following Examples are provided to illustrate but not limit the invention.

All references disclosed herein are incorporated by reference in their entireties.

### EXAMPLES

### Example 1

### Method of Evaluating the NMDA-Induced Current Blocking Properties of the Compounds

The drug “dimebon,” 2,8-dimethyl-5-[2-(6-methylpyridyl-3)ethyl]-2,3,4,5-tetrahydro-1H-pyrido[4,3-b]indole dihydrochloride of the Formula:

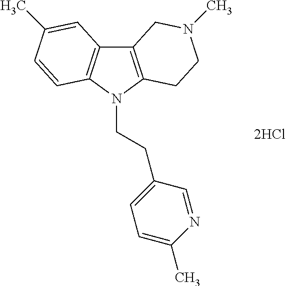

was taken as a representative of the compounds described herein.

Experiments were carried out by the patch clamp method on freshly isolated neurons of a rat brain cortex or on cultured rat hippocampus neurons. Neurons for cultivation were obtained from the hippocampus of neonatal rats (1-2 days) by the method of trypsinization followed by pipetting. Cells suspended in culture medium were placed in 3 mL quantities into the wells of a 6-well planchette (Nunc) or into Petri dishes, in which glasses coated with poly-L-lysine had first been placed. The cell concentration as a rule was 2.5×10⁻⁶-5×10⁻⁶ cell/mL. The culture medium consisted of Eagle's minimum medium and a DME/F12 medium (1:1) supplemented with 10% calf serum, 2 mM glutamine, 50 μg/mL gentamycin, 15 mM glucose and 20 mM KCl, with the pH brought to between 7.0 and 7.4 using NaHCO₃. Planchettes containing cultures were placed in a CO₂— incubator at 37° C. and 100% humidity. Cytosine arabinoside (10-20 μL) was added on the second to third day of cultivation. After 6-7 days of cultivation 1 mg/mL glucose was added to the medium, or the medium was exchanged, depending on the following experiment. The cultured hippocampus neurons were placed in a 0.4 mL working chamber. The working solution had the following composition: 150.0 mM NaCl, 5.0 mM KCl, 2.6 mM CaCl₂, 2.0 mM MgSO₄×7H₂O, 10 mM HEPES, and 15.0 mM Glucose, at pH 7.36.

Transmembrane currents produced by application of NMDA were registered by the patch clamp electrophysiological method in the whole cell configuration. Application of substances was done by the method of rapid superfusion. Currents were registered with the aid of borosilicate microelectrodes (resistance 3.0-4.5 mOhm) filled with the following composition: 100.0 mM KCl, 11.0 mM EGTA, 1.0 mM CaCl₂, 1.0 mM MgCl₂, 10.0 mM HEPES, and 5.0 mM ATP, at pH 7.2. An EPC-9 instrument (HEKA, Germany) was used for registration. Currents were recorded on the hard disk of a Pentium-IV PC using the pulse program, which is also purchased from HEKA. The results were analyzed with the aid of the Pulsefit program (HEKA).

Application of NMDA induced inflow currents in the cultured hippocampus neurons. Dimebon had a blocking effect on currents caused by application of NMDA. The IC50 of dimebon varied from 6.0 to 10 μM, and was an average of 7.7±1.9 μM. MK-801 also caused blockade of NMDA-induced currents. This blockade had a clear “use dependence,” in other words the magnitude of the blocking effect caused by MK-801 was dependent on the preceding effect of the agonist, i.e., NMDA: the blocking effect increased in a series of successive applications of the agonist up to some final value, which was dependent on the concentration of MK-801. 1 μm MK-801 caused blockade of NMDA-induced currents by 70±15%. Preliminary perfusion of neurons with a solution containing dimebon in a concentration of 10 μM caused a decrease of the blocking effect of MK-801 to 40 f 18%. For comparison, the effect of the competing antagonist of the NMDA receptor D-AP5 (D-2-amino-5-phosphonovaleric acid—a selected NMDA receptor antagonist) was investigated for comparison. D-AP5 itself in a dose of 5 μm blocked the NMDA-induced currents by 60-80%. Preliminary application of D-AP5 did not decrease the blocking effect of MK-801.

The results that were obtained are given in Table 1.

TABLE 1
Effect of substances on NMDA-induced currents
in cultured rat hippocampus neurons.

| Substance | Blockade of NMDA-induced currents (%) |
|---|---|
| Dimebon | By 50-70% at 10 μM |
| MK-801 | By 70 ± 15% at 1 μM |
| Dimebon + MK-801 | By 40 ± 18% |
| D-AP5 | By 60-80% at 5 μM |
| D-AP5 + MK-801 | By 75 ± 17% |

The results indicate that dimebon, in spite of the fact that it is itself believed to be an antagonist of NMDA receptors, is capable of reducing the blocking effect of MK-801 on NMDA-induced currents in cultured rat hippocampus neurons. Although the mechanism of the blocking effect of dimebon on NMDA receptors has not yet been established, it does not have the neurotoxic effect that is characteristic for noncompeting blockers of the NMDA receptor ion channel—phencyclidine, MK-801 and ketamine. Based on these new results, it can be suggested that a reduction of the channel-blocking effect of MK-801 (and analogously phencyclidine) on NMDA receptors can lead to a decrease of their psychotomimetic effect and, therefore, to elimination of symptoms characteristic for schizophrenia.

These results indicate that dimebon, along with its previously described properties, can be used for effective treatment of schizophrenia.

### Example 2

### Use of an in Vivo Model to Determine the Ability of Compounds of the Invention to Treat, Prevent and/or Delay the Onset and/or the Development of Schizophrenia

In vivo models of schizophrenia can be used to determine the ability of any of the hydrogenated pyrido[4,3-b]indoles described herein (e.g., dimebon) to treat and/or prevent and/or delay the onset and/or the development of schizophrenia.

One exemplary model for testing the activity of one or more hydrogenated pyrido[4,3-b]indoles described herein to treat and/or prevent and/or delay the onset and/or development of schizophrenia employs phencyclidene, which is chronically administered to the animal (e.g., non-primate (rat) or primate (monkey)), resulting in dysfunctions similar to those seen in schizophrenic humans. See Jentsch et al., 1997, Science 277:953-955 and Piercey et al., 1988, Life Sci. 43(4):375-385). Standard experimental protocols may be employed in this or in other animal models.

### Example 3

### Use of Human Clinical Trials to Determine the Ability of Compounds of the Invention to Treat, Prevent and/or Delay the Onset and/or the Development of Schizophrenia

If desired, any of the hydrogenated pyrido[4,3-b]indoles described herein (e.g., dimebon) can also be tested in humans to determine the ability of the compound to treat, prevent and/or delay the onset and/or the development of schizophrenia. Standard methods can be used for these clinical trials.

In one exemplary method, subjects with schizophrenia are enrolled in a safety, tolerability, pharmacokinetics and pharmacodynamics phase I study of a hydrogenated pyrido[4,3-b]indole using standard protocols. Then a phase II, double-blind randomized controlled trial is performed to determine the efficacy of the hydrogenated pyrido[4,3-b]indole.

### Example 4

### Human Clinical Trials of Combination Therapies of the Invention to Treat, Prevent and/or Delay the Onset and/or the Development of Schizophrenia

A double-blind, placebo-controlled clinical study was conducted to evaluate the effect of a combination therapy of risperidone (3-[2-[4-(6-fluoro-1,2-benzoxazol-3-yl)piperidin-1-yl]ethyl]-2-methyl-6,7,8,9-tetrahydropyrido[2,1-b]pyrimidin-4-one) plus dimebon (2,8-dimethyl-5-(2-(6-methyl-3-pyridyl)ethyl)-2,3,4,5-tetrahydro-1H-pyrido[4,3-b]indole dihydrochloride) for treatment of chronic schizophrenia compared to risperidone treatment alone. The treatment trial was designed as randomized placebo-controlled, double-blind study of 60 patients meeting diagnostic criteria for schizophrenia, paranoid type, and episodic course (DSM IV-295.30).

The study evaluated the effect of switching patients with chronic schizophrenia from the traditional therapy to monotherapy with an atypical antipsychotic agent (risperidone, a serotonin-dopamine blocker) in accordance with clinical indicators, results of neurocognitive tests, neurochemical, neuroimmunological, biochemical, genetic and morphological markers. Risperidone is also known as Rispolept in Russia and is marketed in the United States under the Trade Name Risperdal, and intends the compound 3-[2-[4-(6-fluoro-1,2-benzoxazol-3-yl)piperidin-1-yl]ethyl]-2-methyl-6,7,8,9-tetrahydropyrido[2,1-b]pyrimidin-4-one. The study also evaluated the possible increase of the clinical effect of antipsychotic therapy when dimebon was added to monotherapy with risperidone.

The study included 60 patients with a diagnosis of continuous paranoid and episodic-progressive schizophrenia with a current condition of drug remission or development of drug remission, without regard to the syndrome determining the current mental status. The study did not include patients who were completely resistant to psychotropic therapy. According to DSM-IV, these patients met criteria 295.30, schizophrenia, paranoid type, chronic course. Patients were selected to participate in the study according to the following criteria: (1) inclusion criteria: male patients, 18 year old or older, schizophrenia, paranoid type, and episodic course in remission or partial remission and responders or partial responders to risperidone; and (2) exclusion criteria: excitement, impulsiveness or aggressive behavior (more than moderate), non-responders to antipsychotic treatment including risperidone, having mental disorders other than (or in addition to) schizophrenia, necessity of other treatment and severe acute somatic diseases or decompensated chronic somatic diseases.

In the first stage of the study (2 weeks), patients who provide an informed consent for participation in the study and who met the inclusion/exclusion criteria were switched to monotherapy with risperidone (dose adjustment of risperidone in range from 2 mg to 6 mg per day), and the clinical stability of the patients was evaluated weekly over two weeks. In the second stage (4 weeks), patients who proved susceptible (responders) to the new treatment continued therapy at fixed dosages (about 6 mg risperidone, once daily), and those who were not sensitive to the new treatment (non-responders) were withdrawn from the program and stabilized in routine therapy. Again, clinical stability of the study participants was evaluated weekly for four weeks. In the third stage (8 weeks), after 4 weeks of monotherapy with risperidone, patients were divided into two groups. Group 1 received dimebon, (20 mg three times daily, n=23) in addition to risperidone therapy. Group 2 received a placebo (n=24) in addition to risperidone therapy. Again, clinical stability of the study participants was evaluated weekly for eight weeks. In the fourth stage (follow-up period), 6 months after completion of therapy within the framework of the protocol, the stability of the characteristics achieved in the course of the study were evaluated. In the fourth stage, clinical stability of the study participants was evaluated monthly for six months.

TABLE 1
Study protocol

| First Stage | Second Stage | Third Stage | Fourth Stage |
|---|---|---|---|
| 2 weeks | 4 weeks | 8 weeks | Follow-up visit 6 months |
| | | | after completion of partici- |
| | | | pation in protocol |
| One visit per | One visit per | One visit per | One visit per month |
| week | week | week | |

The study was conducted as follows. The patients were evaluated clinically and by psychopathological (descriptive) methods during every visit. The patients were evaluated according to the psychometric method at the end of the first period, at the end of the second period, after 4 weeks of the third period and at the end of the third period using the following criteria (A) Efficacy assessment scales such as (1) Positive and Negative Syndrome Scale (PANSS scale; Kay, S. R., Fiszbein, A., and Opler, L. A., Schizophrenia Bulletin 13(2):261-76 (1987)); (2) Clinical Global Impression-Severity (CGI-S) and Clinical Global Impression-Improvement (CGI-I); (3) Calgary depression rating scale (Addington, D., Addington, J., and Maticka-Tyndale E., British J. Psych. Suppl. 22:39-44 (1993)); and (4) Negative Symptoms Assessment-16 (NSA-16) (Alphs, L., Summerfelt, A., Lann, H., and Muller, R. J., Psychopharm. Bull. 25:159-163 (1989)); and (B) Safety scales such as (1) the Barnes Akathisia Rating Scale (BARS) (Barnes, T. R., British J. Psych. 154:672-676 (1989)); and (2) the Simpson-Angus Rating Scale (SARS) (Simpson, G. N. and Angus, J. W. S., Acta Psych. Scand. 212(suppl. 44):11-19 (1970)). Neurocognitive functioning methods were used at the end of the second period, after 4 weeks of the third period and at the end of the third period. The neurocognitive assessment tests include (1) working memory tests, including the Wechsler memory scale (a battery of tests for assessing an individual's memory of personal and current information, orientation, mental control, logical memory, digit span, visual memory, and associative learning), subtest V: series A, Wechsler memory scale, subtest V: series B, and Wechsler memory scale, subtest V: sum A and B; (2) associative memory tests such as the Wechsler memory scale, subtest VII; (3) psychomotor speed tests such as the Wechsler test, subtest VII: Symbol coding (4) verbal memory tests such as Text reconstruction; (5) visual-spatial memory tests such as the Benton visual intention test (a test of visual perception and visual memory); (6) attention tests such as the Schulte tables (a test of stability and shifting of voluntary attention), the continuous attention task (CAT) (a test of attention) and the Bourdon test (a test of prolonged attention); and (7) executive functions tests such as the Tower of London (Shallice, T., “Specific impairments of planning,” Phil. Trans. Royal Soc. London, Series B, Biol. Sci. 298(1089):199-209 (1982)), and Wisconsin card sorting test (Berg, E. A., “A simple objective technique for measuring flexibility in thinking,” J. Gen. Psych. 39:15-22 (1948)).

The following biological markers may be evaluated: (1) Neurochemical characteristics such as proteins similar to glutamine synthetase and Cytochrome C oxidase; (2) Neuromorphological characteristics such as ultrastructural studies of lymphocytes and monocytes; (3) Neuroimmunologic characteristics such as cytokines associated with the inflammation reaction (IL-1 beta, IL-2, IFN gamma, tumor necrosis factor); (4) Clinical genetic characteristics such as polymorphic variations of genes for neurotrophic factor of brain and polymorphic variants of serotonin type 2a receptor genes; (5) Molecular biochemical characteristics such as leukocytic elastase activity, a-1 proteinase inhibitor activity, C-reactive protein levels, and levels of antibodies to neuroantigens—factoring the growth of nerves and myelin basic protein; (6) Clinical biochemical characteristics such as level of thrombocytic serotonin, thrombocyte adhesion in column and thrombocyte peak lag time, determination half-life of infuser Tetrahymena pyriformis when incubated with blood serum from patients (“total blood toxicity”) and basic parameters of peroxide oxygenation of lipids in blood of patients.

To assess the significance of changes of scale and test indices inside each group the Wilcoxon Matched Pairs Test (Hodges, J. L. and Lehmann E. L., J. Am. Stat. Assoc. 68(341):151-158 (1973)) was used. To assess the significance of difference in scale scores between groups the Mann-Whitney Test (Mann, H. B. and Whitney, D. R., Ann. Math. Stat. 18:50-60 (1947) and Wilcoxon, F., Biometr. Bull. 1:80-83 (1945)) was used. The results of the Efficacy assessment and the Neurocognitive assessment are tabulated in Tables 2 and 3 respectively

TABLE 2
Efficacy scales indices

| | Dimebon group | | | Placebo group | | |
|---|---|---|---|---|---|---|
| | start | end | Difference | start | end | Difference |
| PANSS total | 78.5 ± 14.1 | 61.9 ± 22.2 | P = 0.008 | 71.8 ± 17.99 | 67.75 ± 16 | P = 0.00054 |
| PANSS | 16.9 ± 4.6 | 12.0 ± 5.9 | P = 0.0049 | 14.75 ± 4.87 | 13.75 ± 3.85 | P = 0.0029 |
| positive | | | | | | |
| PANSS | 22.5 ± 5.15 | 18.48 ± 7.01 | P = 0.03 | 21.3 ± 6.1 | 21 ± 6.1 | ns |
| negative | | | | | | |
| PANSS | 39.1 ± 8.1 | 31.4 ± 11.1 | P = 0.024 | 35.8 ± 9.04 | 33 ± 7.97 | P = 0.0009 |
| general | | | | | | |
| psychopathol | | | | | | |
| % | | 8.14 ± 49.4 | | | 14.4 ± 14.05 | |
| improvement | | | | | | |
| CGI I | | 2.7 ± 1.0 | | | 2.7 ± 0.7 | |
| CGI-S | 4.3 ± 1 | 3.9 ± 1.2 | P = 0.02 | 4.75 ± 0.9 | 4.15 ± 0.67 | P = 0.003 |
| Calgary | 11.6 ± 11.6 | 8.1 ± 9.97 | P = 0.034 | 10.78 ± 14.4 | 9.0 ± 10.98 | ns |
| scale of | | | | | | |
| depression | | | | | | |
| NSA-16 | 64.65 ± 22.8 | 48.5 ± 22.7 | P = 0.008 | 68.0 ± 13.0 | 61.95 ± 13.7 | P = 0.003 |

Table 2 presents the results of the psychometric evaluations of the study participants. The dimebon group demonstrated a positive trend in positive subscale score to the end (p=0.068) in the PANSS scale. The percentage of improvement was comparable between groups, however an improvement was observed in the subgroup of responders (more than 20% of improvement) in the Dimebon group (p=0.07). The CGI-severity and CGI improvement scores were comparable between groups. The total score of Calgary scale of depression were comparable in both groups at the beginning and to the end. The total score of NSA-16 was comparable between groups, however a change of score was observed in the Dimebon group (p=0.036). In the dimebon group, analyses of sum scores of separate blocks of NSA showed favorable difference in speech block at the beginning (p=0.047) and to the end (p=0.01) and in emotions block showed favorable difference only to the end (p=0.07) without any difference at the beginning. The groups had comparable scores of sociability block and general symptoms block at the beginning and towards the end. Significant differences between groups were observed for the following criteria: long pauses before response (p=0.00017), limited volume of speech production (0.018) and slow movement (0.0004) in favor of dimebon. No safety issues were observed in any of the study groups, the safety scales BARS and SARS were comparable.

The results demonstrate that adjunctive dimebon treatment of schizophrenic patients on background risperidone therapy results in a significant reduction in total PANSS score relative to those randomized to placebo. PANSS score is the most commonly used summary measure of positive symptoms, negative symptoms, and general psychopathology in clinical trials of subjects with schizophrenia. Furthermore, analysis of the differences between dimebon and placebo on PANSS positive, as well as on PANSS negative, change scores showed that benefit in both the positive as well as negative symptom domains was observed. The difference in change scores on the NSA-16, comparing dimebon to placebo, supports the utility of dimebon in treating negative symptoms of schizophrenia. The data also suggested a benefit on general psychopathology attributable to dimebon treatment. There were no safety issues in any of the study groups and no differences between the study groups on the BARS and SARS safety scales, reflecting dimebon's safety profile.

TABLE 3
Neurocognitive testing results.

| | Dimebon group | | | Placebo group | | |
|---|---|---|---|---|---|---|
| | start | end | Difference | start | end | Difference |
| Wechsler | 6 ± 1 | 6 ± 0.9 | P = 0.09 | 6 ± 1.17 | 6.5 ± 1 | P = 0.12 |
| memory scale, | | | | | | |
| subtest V: series | | | | | | |
| A | | | | | | |
| Wechsler | 4 ± 1.2 | 4 ± 0.9 | P = 0.55 | 4 ± 1.2 | 4 ± 1.3 | P = 0.03417* |
| memory scale, | | | | | | |
| subtestV: series | | | | | | |
| B, | | | | | | |
| Wechsler | 10 ± 1.7 | 9.5 ± 2.7 | P = 0.45 | 10 ± 2.1 | 10.5 ± 2 | P = 0.0005* |
| memory scale, | | | | | | |
| subtest V: sum | | | | | | |
| A and B | | | | | | |
| Wechsler | 9 ± 2.3 | 8 ± 2.3 | P = 0.42 | 9 ± 3.1 | 9.5 ± 2.7 | P = 0.0185* |
| memory scale, | | | | | | |
| subtest V: sum | | | | | | |
| A and B, (T- | | | | | | |
| scores) | | | | | | |
| Wechsler | 14 ± 4 | 17 ± 2.9 | P = 0.02* | 15 ± 2.3 | 16 ± 5.3 | P = 0.52 |
| memory scale, | | | | | | |
| subtest VII: sum | | | | | | |
| simple | | | | | | |
| Wechsler | 3 ± 2.7 | 4.5 ± 3.34 | P = 0.06 | 4.5 ± 3.28 | 5 ± 4.24 | P = 0.98 |
| memory scale, | | | | | | |
| subtest VII: sum | | | | | | |
| comlex | | | | | | |
| Wechsler test, | 5 ± 1.8 | 6 ± 1.4 | P = 0.02* | 6 ± 2.2 | 7 ± 2.8 | P = 0.36 |
| subtest VII: | | | | | | |
| Symbol coding | | | | | | |
| Text 1 | 5 ± 2.3 | 7 ± 2.6 | P = 0.007* | 6 ± 3.3 | 7.5 ± 4.49 | P = 0.0004* |
| Reconstruction | | | | | | |
| 1. quantity of | | | | | | |
| elements | | | | | | |
| Text 2 | 7 ± 2.7 | 9 ± 3.0 | P = 0.009* | 8 ± 3.2 | 11.5 ± 3.8 | P = 0.0002* |
| Reconstruction 1 | | | | | | |
| quantity of | | | | | | |
| elements | | | | | | |
| Benton test: sum | 5 ± 1.7 | 6 ± 2.21 | P = 0.038* | 6 ± 2.3 | 6.5 ± 2.26 | 0.9 |
| reconst | | | | | | |
| Benton test: sum | 8 ± 3.8 | 6.5 ± 4.4 | P = 0.21 | 6 ± 4 | 5 ± 4 | P = 0.9 |
| error | | | | | | |
| Schulte tables: | 160 ± 105 | 166 ± 64 | P = 0.89 | 169 ± 61.4 | 132 ± 108 | P = 0.297 |
| sum time | | | | | | |
| CAT: correct | 35 ± 8.5 | 37 ± 8.2 | P = 0.47 | 36 ± 8.3 | 38 ± 12 | P = 0.6 |
| answer account | | | | | | |
| CAT: reaction | 606.74± | 630.17 ± 195.55 | P = 0.136 | 738.9 ± 264.27 | 666.9 ± 195.3 | P = 0.198 |
| time | | | | | | |
| Bourdohn test: | 0.975 ± 0.03 | 0.986 ± 0.013 | P = 0.005* | 0.986 ± 0.002 | 0.989 ± 0.025 | P = 0.550 |
| attention | | | | | | |
| concentration | | | | | | |
| Bourdohn test: | 0.01 ± 0.01 | 0.009 ± 0.006 | P = 0.002* | 0.01 ± 0.009 | 0.01 ± 0.01 | P = 0.06 |
| attention | | | | | | |
| stability | | | | | | |
| Bourdohn | 399 ± 123 | 497 ± 170 | P = 0.014* | 454 ± 167 | 515 ± 224 | P = 0.055 |
| test: attention | | | | | | |
| productivity | | | | | | |
| Tower of | 2 ± 1.1 | 3.26 | P = 0.04* | 3 ± 2.2 | 3 ± 2.9 | P = 0.148 |
| London - correct | | | | | | |
| score | | | | | | |
| Tower of | 46 ± 25 | 34 ± 19 | P = 0.01* | 38 ± 20 | 35 ± 31 | P = 0.43 |
| London -move | | | | | | |
| score | | | | | | |
| Tower of | 1.5 ± 3 | 0 ± 1.5 | P = 0.007* | 1 ± 2.4 | 1 ± 3 | P = 0.838 |
| London - sum | | | | | | |
| total time > 60 | | | | | | |
| sec | | | | | | |
| Tower of | 298 ± 227 | 234 ± 115 | P = 0.02* | 251 ± 207 | 234 ± 229 | P = 0.058 |
| London - | | | | | | |
| execution time | | | | | | |
| Wisconsin CS | 79 ± 16 | 78 ± 14 | P = 0.463 | 76 ± 17 | 74 ± 10 | P = 0.888 |
| test: total correct | | | | | | |
| responses | | | | | | |
| Wisconsin CS | 4 ± 1.7 | 5 ± 1.9 | P = 0.919 | 6 ± 2.9 | 6 ± 2 | P = 0.865 |
| test: categories | | | | | | |
| completed | | | | | | |
| Wisconsin CS | 43 ± 6.8 | 43 ± 8.1 | P = 0.27 | 43 ± 9.4 | 44 ± 9.1 | P = 0.027* |
| test - total | | | | | | |
| error - T score | | | | | | |
| Wisconsin CS | 43 ± 8.38 | 45 ± 5.53 | P = 0.132 | 47 ± 6.1 | 43 ± 9.2 | P = 0.833 |
| test - | | | | | | |
| perseverative | | | | | | |
| responses, T | | | | | | |
| score | | | | | | |
| Wisconsin CS | 43 ± 8.21 | 44 ± 5.6 | P = 0.046* | 47 ± 6 | 44 ± 9.6 | P = 0.906 |
| test | | | | | | |
| perseverative | | | | | | |
| errors, T score | | | | | | |
| Wisconsin CS | 41 ± 7.9 | 43 ± 9.6 | P = 0.869 | 41 ± 10.3 | 47 ± 8.7 | P = 0.010* |
| test - | | | | | | |
| nonperseverative | | | | | | |
| errors, T score | | | | | | |
| Wisconsin CS | 43 ± 5.9 | 44 ± 6.7 | P = 0.271 | 45 ± 8.7 | 46 ± 12 | P = 0.036* |
| test - conceptual | | | | | | |
| level responses, | | | | | | |
| T score | | | | | | |

Table 3 presents the results of the neurocognitive evaluations of the study participants. Difference values marked with an asterisk (“*”) in Table 3 indicate test results with a statistically significant difference from start to finish of the clinical trial as determined by either the Wilcoxon Matched Pairs Test or the Mann-Whitney Test as described herein. A few differences in neurocognitive indices were observed between the groups both at the beginning and to the end of the trial. No deteriorations were observed during any testing. Significant improvement was observed in both groups in verbal semantic memory: (1) The dimebon group demonstrated significant improvement in verbal associative memory, psychomotor speed, visual-spatial memory and number aspects of executive functioning—planning, purposeful activity and control upon the results of activity (perseverative errors), as shown by the results of the Wechsler Memory Scale Subtest VII, the Text Reconstruction test, the Benton test, the Bourdohn test, and the Tower of London test; (2) The placebo group demonstrated significant improvement in working memory and control upon the results of activity (nonperseverative errors), as shown by the results of the Wechsler Memory Scale Subtest V, the Text Reconstruction test, and the Wisconsin CS test. The placebo-controlled, double-blind portion of the study lasted only eight weeks, which would be considered short for a trial of a putative enhancer of cognition in schizophrenia patients. Thus, these results suggest the potential for dimebon to provide a cognitive benefit in this population when studied for a longer duration, particularly in the memory and executive function domains that are known to be particularly affected in schizophrenia.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

All references, publications, patents, and patent applications disclosed herein are hereby incorporated herein by reference in their entireties.

1. A method of (a) treating schizophrenia in an individual in need thereof; (b) slowing the progression of schizophrenia in an individual who has been diagnosed with schizophrenia; or (c) preventing or delaying development of schizophrenia in an individual who is at risk of developing schizophrenia, the method comprising administering to the individual an effective amount of a combination therapy comprising dimebon and an antipsychotic.

2. The method of claim 1, wherein the antipsychotic is an atypical antipsychotic.

3. The method of claim 2, wherein the atypical antipsychotic is selected from the group consisting of risperidone, clozapine, N-desmethylclozapine, olanzapine, quetiapine, perospirone, ziprasidone, olanzapine/fluoxetine (marketed as Symbyax™), aripiprazole, paliperidone, sertindole, zotepine, amisulpride, bifeprunox, asenapine, melperone, abaperidone, blonanserin, iloperidone, lurasidone, ocaperidone, QF-2400B, SB-773812, ITI-007, and YKP-1358.

4. The method of claim 3, wherein the atypical antipsychotic is risperidone.

5. The method of claim 1, wherein administration of dimebon enhances the therapeutic effect of the antipsychotic compared to administration of the antipsychotic in the absence of dimebon.

6. The method of claim 1, wherein the antipsychotic is administered in a dosage amount that is less than that required for the antipsychotic as an individual therapy to elicit a comparable therapeutic effect.

7. A pharmaceutically acceptable composition comprising dimebon and an antipsychotic.

8. The composition of claim 7, wherein the antipsychotic is an atypical antipsychotic.

9. The composition of claim 8, wherein the atypical antipsychotic is selected from the group consisting of risperidone, clozapine, N-desmethylclozapine, olanzapine, quetiapine, perospirone, ziprasidone, olanzapine/fluoxetine (marketed as Symbyax™), aripiprazole, paliperidone, sertindole, zotepine, amisulpride, bifeprunox, asenapine, melperone, abaperidone, blonanserin, iloperidone, lurasidone, ocaperidone, QF-2400B, SB-773812, ITI-007, and YKP-1358.

10. The composition of claim 9, wherein the atypical antipsychotic is risperidone.

11. The composition of claim 7, wherein dimebon and the antipsychotic are in a single unit dosage form.

12. A kit comprising: (a) dimebon; (b) an antipsychotic; and (c) instructions for use of in the treatment, prevention, slowing the progression or delaying the onset and/or development of schizophrenia.

13. The kit of claim 12, wherein the antipsychotic is an atypical antipsychotic.

14. The kit of claim 13, wherein the atypical antipsychotic is selected from the group consisting of risperidone, clozapine, N-desmethylclozapine, olanzapine, quetiapine, perospirone, ziprasidone, olanzapine/fluoxetine (marketed as Symbyax™), aripiprazole, paliperidone, sertindole, zotepine, amisulpride, bifeprunox, asenapine, melperone, abaperidone, blonanserin, iloperidone, lurasidone, ocaperidone, QF-2400B, SB-773812, ITI-007, and YKP-1358.

15. The kit of claim 14, wherein the atypical antipsychotic is risperidone.

16. A method of enhancing an individual's response to an antipsychotic comprising administering dimebon in connection with the antipsychotic, wherein the individual has or is suspected of having schizophrenia.

17. The method of claim 16, wherein the antipsychotic is an atypical antipsychotic.

18. The method of claim 17, wherein the atypical antipsychotic is selected from the group consisting of risperidone, clozapine, N-desmethylclozapine, olanzapine, quetiapine, perospirone, ziprasidone, olanzapine/fluoxetine (marketed as Symbyax™), aripiprazole, paliperidone, sertindole, zotepine, amisulpride, bifeprunox, asenapine, melperone, abaperidone, blonanserin, iloperidone, lurasidone, ocaperidone, QF-2400B, SB-773812, ITI-007, and YKP-1358.

19. The method of claim 18, wherein the atypical antipsychotic is risperidone.

20. A method of treating schizophrenia in an individual in need thereof comprising administering to an individual a combination therapy comprising dimebon and an antipsychotic wherein the combination therapy is administered in an amount effective to improve a cognitive symptom of schizophrenia and wherein the combination therapy elicits cognitive improvement to a greater extent than use of the antipsychotic in the absence of dimebon.

21. The method of claim 20, wherein the antipsychotic is an atypical antipsychotic.

22. The method of claim 21, wherein the atypical antipsychotic is selected from the group consisting of risperidone, clozapine, N-desmethylclozapine, olanzapine, quetiapine, perospirone, ziprasidone, olanzapine/fluoxetine (marketed as Symbyax™), aripiprazole, paliperidone, sertindole, zotepine, amisulpride, bifeprunox, asenapine, melperone, abaperidone, blonanserin, iloperidone, lurasidone, ocaperidone, QF-2400B, SB-773812, ITI-007, and YKP-1358.

23. The method of claim 22, wherein the atypical antipsychotic is risperidone.
